SEQUENCE LISTING (58 sequences)

SEQ ID NO: 1 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 2 - protein, 19 aa, Homo sapiens
EYASPGKATEVRVTVLRQA

SEQ ID NO: 3 - protein, 18 aa, Homo sapiens
RQADSQVTEVCAATYMMG

SEQ ID NO: 4 - protein, 39 aa, Homo sapiens
GNELTFLDDSICTGTSSGNQVNFHMSVVRARRNDSGTYL

SEQ ID NO: 5 - protein, 21 aa, Homo sapiens
KVELMYPPPYYLGIGNGTQIY

SEQ ID NO: 6 - protein, 18 aa, Measles morbillivirus
KLLSLIKGVIVHRLEGVE

SEQ ID NO: 7 - protein, 4 aa, synthetic construct
GPSL

SEQ ID NO: 8 - protein, 41 aa, synthetic construct
KLLSLIKGVIVHRLEGVEGPSLEYASPGKATEVRVTVLRQA

SEQ ID NO: 9 - protein, 40 aa, synthetic construct
KLLSLIKGVIVHRLEGVEGPSLRQADSQVTEVCAATYMMG

SEQ ID NO: 10 - protein, 61 aa, synthetic construct
KLLSLIKGVIVHRLEGVEGPSLGNELTFLDDSICTGTSSGNQVNFHMSVVRARRNDSGTYL

SEQ ID NO: 11 - protein, 43 aa, synthetic construct
KLLSLIKGVIVHRLEGVEGPSLKVELMYPPPYYLGIGNGTQIY

SEQ ID NO: 12 - protein, 19 aa, synthetic construct
AQRLVTVRVETAKGPSAYE

SEQ ID NO: 13 - protein, 18 aa, synthetic construct
GMMYTAACVETVQSDAQR

SEQ ID NO: 14 - protein, 39 aa, synthetic construct
LYTGSDNRRARVVSMHFNVQNGSSTGTCISDDLFTLENG

SEQ ID NO: 15 - protein, 21 aa, synthetic construct
YIQTGNGIGLYYPPPYMLEVK

SEQ ID NO: 16 - protein, 41 aa, synthetic construct
KLLSLIKGVIVHRLEGVEGPSLAQRLVTVRVETAKGPSAYE

SEQ ID NO: 17 - protein, 40 aa, synthetic construct
KLLSLIKGVIVHRLEGVEGPSLGMMYTAACVETVQSDAQR

SEQ ID NO: 18 - protein, 61 aa, synthetic construct
KLLSLIKGVIVHRLEGVEGPSLLYTGSDNRRARVVSMHFNVQNGSSTGTCISDDLFTLENG

SEQ ID NO: 19 - protein, 43 aa, synthetic construct
KLLSLIKGVIVHRLEGVEGPSLYIQTGNGIGLYYPPPYMLEVK

SEQ ID NO: 20 - protein, 19 aa, synthetic construct
NSVDDALINSTIYSYFPSV

SEQ ID NO: 21 - protein, 17 aa, synthetic construct
PGINGKAIHLVNNQSSE

SEQ ID NO: 22 - protein, 15 aa, synthetic construct
QYIKANSKFIGITEL

SEQ ID NO: 23 - protein, 21 aa, synthetic construct
FNNFTVSFWLRVPKVSASHLE

SEQ ID NO: 24 - protein, 15 aa, synthetic construct
LSEIKGVIVHRLEGV

SEQ ID NO: 25 - protein, 15 aa, synthetic construct
FFLLTRILTIPQSLN

SEQ ID NO: 26 - protein, 20 aa, synthetic construct
TCGVGVRVRSRVNAANKKPE

SEQ ID NO: 27 - protein, 19 aa, synthetic construct
  SITE 1: note = acetylation of N-terminus
EYASPGKATEVRVTVLRQA

SEQ ID NO: 28 - protein, 18 aa, synthetic construct
  SITE 1: note = acetylation of N-terminus
RQADSQVTEVCAATYMMG

SEQ ID NO: 29 - protein, 39 aa, synthetic construct; identical to SEQ ID NO: 4
  SITE 1: note = acetylation of N-terminus

SEQ ID NO: 30 - protein, 21 aa, synthetic construct
  SITE 1: note = acetylation of N-terminus
KVELMYPPPYYLGIGNGTQIY

SEQ ID NO: 31 - protein, 19 aa, synthetic construct
  SITE 1: note = acetylation of N-terminus
AQRLVTVRVETAKGPSAYE

SEQ ID NO: 32 - protein, 18 aa, synthetic construct
  SITE 1: note = acetylation of N-terminus
GMMYTAACVETVQSDAQR

SEQ ID NO: 33 - protein, 39 aa, synthetic construct; identical to SEQ ID NO: 14
  SITE 1: note = acetylation of N-terminus

SEQ ID NO: 34 - protein, 21 aa, synthetic construct
  SITE 1: note = acetylation of N-terminus
YIQTGNGIGLYYPPPYMLEVK

SEQ ID NO: 35 - protein, 19 aa, synthetic construct
  SITE 1..19: note = D-amino acids
EYASPGKATEVRVTVLRQA

SEQ ID NO: 36 - protein, 18 aa, synthetic construct
  SITE 1..18: note = D-amino acids
RQADSQVTEVCAATYMMG

SEQ ID NO: 37 - protein, 39 aa, synthetic construct; identical to SEQ ID NO: 4
  SITE 1..39: note = D-amino acids

SEQ ID NO: 38 - protein, 21 aa, synthetic construct
  SITE 1..21: note = D-amino acids
KVELMYPPPYYLGIGNGTQIY

SEQ ID NO: 39 - protein, 19 aa, synthetic construct
  SITE 1: note = acetylation of N-terminus
  SITE 1..19: note = D-amino acids
EYASPGKATEVRVTVLRQA

SEQ ID NO: 40 - protein, 18 aa, synthetic construct
  SITE 1: note = acetylation of N-terminus
  SITE 1..18: note = D-amino acids
RQADSQVTEVCAATYMMG

SEQ ID NO: 41 - protein, 39 aa, synthetic construct; identical to SEQ ID NO: 4
  SITE 1: note = acetylation of N-terminus
  SITE 1..39: note = D-amino acids

SEQ ID NO: 42 - protein, 21 aa, synthetic construct
  SITE 1: note = acetylation of N-terminus
  SITE 1..21: note = D-amino acids
KVELMYPPPYYLGIGNGTQIY

SEQ ID NO: 43 - protein, 41 aa, synthetic construct; identical to SEQ ID NO: 8
  SITE 23..41: note = D-amino acids

SEQ ID NO: 44 - protein, 40 aa, synthetic construct; identical to SEQ ID NO: 9
  SITE 23..40: note = D-amino acids

SEQ ID NO: 45 - protein, 61 aa, synthetic construct; identical to SEQ ID NO: 10
  SITE 23..61: note = D-amino acids

SEQ ID NO: 46 - protein, 43 aa, synthetic construct; identical to SEQ ID NO: 11
  SITE 23..43: note = D-amino acids

SEQ ID NO: 47 - protein, 19 aa, synthetic construct
  SITE 1..19: note = D-amino acids
AQRLVTVRVETAKGPSAYE

SEQ ID NO: 48 - protein, 18 aa, synthetic construct
  SITE 1..18: note = D-amino acids
GMMYTAACVETVQSDAQR

SEQ ID NO: 49 - protein, 39 aa, synthetic construct; identical to SEQ ID NO: 14
  SITE 1..39: note = D-amino acids

SEQ ID NO: 50 - protein, 21 aa, synthetic construct
  SITE 1..21: note = D-amino acids
YIQTGNGIGLYYPPPYMLEVK

SEQ ID NO: 51 - protein, 19 aa, synthetic construct
  SITE 1: note = acetylation of N-terminus
  SITE 1..19: note = D-amino acids
AQRLVTVRVETAKGPSAYE

SEQ ID NO: 52 - protein, 18 aa, synthetic construct
  SITE 1: note = acetylation of N-terminus
  SITE 1..18: note = D-amino acids
GMMYTAACVETVQSDAQR

SEQ ID NO: 53 - protein, 39 aa, synthetic construct; identical to SEQ ID NO: 14
  SITE 1: note = acetylation of N-terminus
  SITE 1..39: note = D-amino acids

SEQ ID NO: 54 - protein, 21 aa, synthetic construct
  SITE 1: note = acetylation of N-terminus
  SITE 1..21: note = D-amino acids
YIQTGNGIGLYYPPPYMLEVK

SEQ ID NO: 55 - protein, 41 aa, synthetic construct; identical to SEQ ID NO: 16
  SITE 23..41: note = D-amino acids

SEQ ID NO: 56 - protein, 40 aa, synthetic construct; identical to SEQ ID NO: 17
  SITE 23..40: note = D-amino acids

SEQ ID NO: 57 - protein, 61 aa, synthetic construct; identical to SEQ ID NO: 18
  SITE 23..61: note = D-amino acids

SEQ ID NO: 58 - protein, 43 aa, synthetic construct; identical to SEQ ID NO: 19
  SITE 23..43: note = D-amino acids

(19) United States
(12) Patent Application Publication
(10) Pub. No.: US 2026/0007730 A1
(43) Pub. Date: Jan. 8, 2026

(54) HUMAN CTLA-4 PEPTIDE VACCINES AND USES THEREOF

(71) Applicant: Ohio State Innovation Foundation, Columbus, OH (US)

(72) Inventor: Pravin KAUMAYA, Westerville, OH (US)

(21) Appl. No.: 18/857,573

(22) PCT Filed: Apr. 21, 2023
(86) PCT No.: PCT/US2023/066090

§ 371 Date: Oct. 17, 2024

Related U.S. Application Data
(60) Provisional application No. 63/333,419, filed on Apr. 21, 2022.

(51) Int. Cl. A61K 39/00; A61K 39/165; A61P 37/04; C07K 14/005; C07K 14/705

(52) U.S. Cl. CPC A61K 39/001111; A61K 39/165; A61P 37/04; C07K 14/005; C07K 14/70521; A61K 2039/585; C07K 2319/00; C12N 2760/18422; C12N 2760/18434; C12N 2760/18471

(57) ABSTRACT

Disclosed are compositions related to synthetic CTLA-4 peptides, chimeric CTLA-4 peptides, anti-CTLA-4 antibodies and methods of treating cancers, autoimmune diseases, and Alzheimer's disease using said peptides or antibodies.

### CROSS-REFERENCE TO RELATED APPLICATIONS

This application is a U.S. National Stage application filed under 35 U.S.C. § 371 of PCT/US2023/066090, filed on Apr. 21, 2023, which claims the benefit of U.S. Provisional Application 63/333,419, filed Apr. 21, 2022, each of which is incorporated herein by reference in their entirety.

### REFERENCE TO SEQUENCE LISTING

The sequence listing submitted on Jun. 16, 2025, as an .XML file entitled “103361-260US1.XML” created on Jun. 16, 2025, and having a file size 67,674 bytes is hereby incorporated by reference pursuant to 37 C.F.R. § 1.52 (e) (5).

### I. BACKGROUND

Cancer is now the primary cause of death in developed countries and world-wide. The financial burden of this disease, and more importantly, the suffering it causes, is immense. There is an obvious and urgent need to speed the development and application of new, more efficacious anti-cancer therapies. The field of oncology is vast and comprises several indications, including some rare/orphan forms. Although oncology continues to be one of the most active areas in terms of drug development, there is still a significant unmet need.

Recent advances in cancer immunology have documented the importance of T cell-mediated anti-tumor immunity against human cancers, and inhibitory receptors expressed by T cells have become important targets for cancer immunotherapy. Checkpoint inhibitors act by blocking the pathways that inhibit immune cell activation and stimulate immune responses against tumor cells, have been greatly successful in the treatment of cancer either as monotherapy or in combination, have revolutionized the treatment landscape of cancer patients. The success of immune checkpoint inhibitors (ICIs), notably anti-cytotoxic T lymphocyte associated antigen-4 (CTLA-4) as well as inhibitors of CTLA-4, programmed death 1 (PD-1), and programmed death ligand-1 (PD-L1), has revolutionized treatment options for solid tumors. The advent of immune checkpoint receptors has been one of the most fruitful, stimulating, and studied strategies in immune-oncology and vaccine immunotherapy. Currently, several immune checkpoint inhibitors, mostly monoclonal antibodies, have shown significant results albeit major drawbacks exist in that only 10-20% of patient responding and induction of severe immune-related adverse effects. Small molecules are being studied extensively as alternative approaches to mAbs.

Anti-PD-1 agents (nivolumab, pembrolizumab, cemiplimab) and anti-PD-L1 agents (atezolizumab, avelumab, durvalumab) were developed later. These agents have been approved for use in multiple solid and hematologic malignancies. Improved treatment outcomes and durable responses have been observed after discontinuation of therapy, however their efficacy was limited to a small number of patients. Monoclonal antibodies targeting immunologic checkpoints and especially the PD-1/PD-L1 axis provided spectacular results in cancer therapy in the recent years. Despite their proven utility, antibodies have specific drawbacks as therapeutics, including poor tissue/tumor penetrance which may be especially pertinent when targeting the PD-1: PD-L1 signaling pathway

Checkpoint blockades turn on a new paradigm shift in immunotherapy for cancer. However, a lot of cancer patients failed to respond to the CTLA-4 checkpoint blockades. What are needed are new CTLA-4 checkpoint inhibitors for the treatment of cancer, viral infections, autoimmune diseases and Alzheimer's disease.

### II. SUMMARY

Disclosed are methods and compositions related to synthetic and/or chimeric cytotoxic T-lymphocyte-associated protein 4 (CTLA-4) peptides.

In one aspect, disclosed herein are cytotoxic T-lymphocyte-associated protein 4 (CTLA-4) chimeric peptides for stimulating an immune response to a CTLA-4 protein comprising one or more CTLA-4 B cell epitopes, a T helper (Th) epitope (for example, a measles virus fusion protein peptide KLLSLIKGVIVHRLEGVE as set forth in (SEQ ID NO: 6)), and a linker (such as, for example, SEQ ID NO: 7) joining the CTLA-4 B cell epitope to the Th epitope, wherein the one or more CTLA-4 B cell epitopes consist of a sequence selected from the group consisting of SEQ ID NO:2, SEQ ID NO:3, SEQ ID NO:4, and SEQ ID NO:5 or the D enantiomer of the disclosed sequences as set forth in SEQ ID NO: 35, SEQ ID: 36, SEQ ID NO: 37, and SEQ ID NO: 38, respectively. For example, disclosed herein are chimeric peptides of any preceding aspect, wherein the peptide comprises the amino acid sequence as set forth in SEQ ID NO: 8, SEQ ID NO: 9, SEQ ID NO: 10, SEQ ID NO:11, SEQ ID NO: 43, SEQ ID NO: 44, SEQ ID NO: 45, or SEQ ID NO: 46.

Also disclosed herein are cytotoxic T-lymphocyte-associated protein 4 (CTLA-4) chimeric peptides for stimulating an immune response to a CTLA-4 protein comprising the D-enantiomer of one or more CTLA-4 B cell epitopes SEQ ID NO:2, SEQ ID NO:3, SEQ ID NO: 4, and SEQ ID NO:5 as set forth in SEQ ID NO: 35, SEQ ID: 36, SEQ ID NO: 37, and SEQ ID NO: 38, respectively.

In one aspect, disclosed herein are synthetic peptides of any preceding aspect, wherein the peptide is acetylated as set forth in SEQ ID NO: 27, SEQ ID NO: 28, SEQ ID NO: 29, SEQ ID NO: 30, SEQ ID NO: 39, SEQ ID NO: 40, SEQ ID NO: 41, and SEQ ID NO: 42.

In one aspect, disclosed herein are synthetic CTLA-4peptides for stimulating an immune response to a CTLA-4 protein comprising one or more of the sequences as set forth in SEQ ID NO: 12, SEQ ID NO: 13, SEQ ID NO: 14, or SEQ ID NO: 15 including the D enantiomer of the disclosed sequences as set forth in SEQ ID NO: 47, SEQ ID: 48, SEQ ID NO: 49, and SEQ ID NO: 50, respectively. In one aspect, the synthetic peptide can be acetylated as set forth in SEQ ID NO: 31, SEQ ID NO: 32, SEQ ID NO: 33, SEQ ID NO: 34, SEQ ID NO: 51, SEQ ID NO: 52, SEQ ID NO: 53, and SEQ ID NO: 54.

Also disclosed herein are chimeric peptides comprising the synthetic peptide of any preceding aspect, further comprising a Th epitope (for example, a measles virus fusion protein peptide such as SEQ ID NO: 6), and a linker (such as, for example, SEQ ID NO: 7) joining the synthetic CTLA-4 peptide to the Th epitope.

Also disclosed herein are chimeric peptides of any preceding aspect, wherein the peptide comprises the amino acid sequence as set forth in SEQ ID NO: 16, SEQ ID NO: 17, SEQ ID NO: 18, SEQ ID NO: 19, SEQ ID NO: 55, SEQ ID NO: 56, SEQ ID NO: 57, and SEQ ID NO: 58.

In one aspect, disclosed herein are pharmaceutical compositions comprising one or more chimeric or synthetic peptides of any preceding and a pharmaceutically acceptable vehicle.

In one aspect, disclosed herein are methods of treating a cancer, Alzheimer's disease, or autoimmune disease in a subject comprising administering to the subject any of the peptides or compositions of any preceding aspect.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, 1C, ID, 1E, and 1F show rabbit response and identification of four B-cell epitope sequences of human CTLA-4 peptides. FIG. 1A shows the amino acid sequences of human CTLA-4, the epitopes of 59-77, 75-92, 92-114 and 130-150 peptides were chosen for investigation. FIG. 1B shows the secondary structure of the sequences of human PD-L1 epitopes as modelled by PyMOL. FIG. 1C shows the structure of the CTLA-4/CD80 complex adapted from PMID: 28484017 and PMID: 28978021, key amino acids involved in the interaction between human CTLA-4 and human CD80 are illustrated. FIG. 1D shows a scheme of immunization with MVF-CTLA-4 B-cell epitopes on New Zealand White rabbits. Rabbits were immunized with 1 mg of each MVF-peptide immunogens dissolved in dd H2O emulsified (1:1) in Montanide ISA 720 vehicle. The rabbits were boosted with the same doses with 3 weeks apart. Blood was collected via the central auricular artery in rabbits. And the terminal sera were collected at 3Y+3 which is 3 weeks after the last immunization. FIG. 1E shows the immunogenicity of MVF-CTLA-4 B-cell epitopes were evaluated by ELISA. The 96-well microplate was coated with 200 ng/well peptide. Titers are defined as the highest dilution of sera with an absorbance value no less than 0.2 after subtracting the blank. FIG. 1F shows immunized rabbits' sera recombinant protein activity against human CTLA-4.

FIGS. 2A, 2B, 2C, and 2D show the immunogenicity and Antigenicity CTLA-4 Peptide Vaccines in BALB/c mice challenged with CT26, 4T1 and D2F2 Tumor Cell Lines. FIG. 2A shows 6-8 weeks old BALB/c mice were vaccinated with MVF-CTLA-4 (59), MVF-CTLA-4 (75), MVF-CTLA-4 (92) or MVF-CTLA-4 (130) for 3 weeks interval. 0.1 mg peptide cancer vaccine mixed with ISA 720 (1:1) used per mouse. Mice were boosted with the designed doses for every 3 weeks intervals. Blood was collected weekly for monitoring antibody titers. After 2 weeks of the third time immunization (3Y), mice were challenged with 10⁵ per mouse CT26, 10⁵ per mouse 4T1 or 2×10⁵ per mouse D2F2 cancer cells. After tumor challenge, the positive control group, we treat the mice with anti-mouse CTLA-4 antibody (clone 9H10) twice a week for up to three weeks, and the negative control group was treated with PBS. FIG. 2B shows the immunogenicity of mice from different groups are showed in the table and bar graph figure as indicated. The highest dilution at the cutoff absorbance 0.2 was determined as the antibody titer. FIG. 2C shows the antibodies isotypes from four CTLA-4 peptide vaccine immunized BALB/c as shown in the graph. The majority of subtype is IgG1 range from 29% to 65%, followed by IgG2a (14% to 29%) and IgG2b (7% to 39%). FIG. 2D shows the anti-CTLA-4 antibody recombinant protein activities against mice sera, pre-immunized sera served as negative control. The plate coated with serial diluted recombinant human CTLA-4 His start from 2.5 μg/ml, concentration as indicated in the figure. The mice sera at 3Y+1 were used as 1:50 dilution.

FIGS. 3A, 3B, 3C, 3D, and 3E show CTLA-4 Peptide Vaccines in CT26-BALB/c Tumor Model. FIG. 3A shows 6-8 weeks old BALB/c mice were vaccinated with MVF-CTLA-4 (59), MVF-CTLA-4 (75), MVF-CTLA-4 (92) or MVF-CTLA-4 (130) for 3 weeks boosted with the designed doses for every 3 weeks intervals. Blood was collected weekly for monitoring antibody titers. After 2 weeks of the third time immunization (3Y), mice were challenged with 10⁵ per mouse CT26. After tumor challenge, the positive control group, we treat the mice with anti-mouse CTLA-4 antibody (clone 9H10) twice a week for up to three weeks, and the negative control group was treated with PBS. Tumor volume was calculated as: Tumor volume (LWW)=(Length×Width×Width)/2. FIG. 3B shows the mean value of tumor growths in BALB/c mice were vaccinated with MVF-CTLA-4 (59), MVF-CTLA-4 (75), MVF-CTLA-4 (92) or MVF-CTLA-4 (130), PBS as negative control and anti-mouse CTLA-4 antibody (clone 9H10) as positive control. Two-way ANOVA was used to analyze the whole curves of tumor growth, which shows significant difference with p<0.01. FIG. 3C shows individual mouse CT26 tumor growths in each group of mice were vaccinated with MVF-CTLA-4 (59), MVF-CTLA-4 (75), MVF-CTLA-4 (92) or MVF-CTLA-4 (130), PBS as negative control and anti-mouse CTLA-4 antibody (clone 9H10) as positive control. FIG. 3D shows plots of tumor volume LWW at day 14 for each of the four treatment immunized groups together with PBS control and mAb (9H10) groups; one-way ANOVA was used to analysis multiple groups comparison, which indicated both of p<0.01. Between groups versus with PBS group, mice in mAb (9H10), CTLA-4 (59), CTLA-4 (92) and CTLA-4 (130) group with significantly smaller tumor size versus with PBS group. Plots of tumor volume LWW at day 16 for each of the four treatment immunized groups together with PBS control and mAb (9H10) groups; one-way ANOVA was used to analysis multiple groups comparison, which indicated both of p<0.01. Between groups versus with PBS group, mice in mAb (9H10), CTLA-4 (59), CTLA-4 (92) and CTLA-4 (130) group with significantly smaller tumor size versus with PBS group. ** indicates p<0.01, * indicates p<0.05, ns indicates no significant difference. FIG. 3E shows 6-8 weeks old BALB/c mice were vaccinated with MVF-CTLA-4 (59), MVF-CTLA-4 (75), MVF-CTLA-4 (92) or MVF-CTLA-4 (130) for 3 weeks interval with two boost. Mice were challenged with 10⁵ per mouse CT26 tumor cells. After tumor challenge, the positive control group, we treat the mice with anti-mouse CTLA-4 antibody (clone 9H10) twice a week for up to three weeks, and the negative control group was treated with PBS. The survival curves Log-rank test for multiple groups p<0.01. Between groups comparison of each individual group versus PBS group p<0.01 as indicated with **. ** indicates p<0.01;

FIGS. 4A, 4B, 4C, 4D, and 4E show CTLA-4 Peptide Vaccines in 4T1-BALB/c Tumor Model. FIG. 4A shows 6-8 weeks old BALB/c mice were vaccinated with MVF-CTLA-4 (59), MVF-CTLA-4 (75), MVF-CTLA-4 (92) or MVF-CTLA-4 (130) for 3 weeks boosted with the designed doses for every 3 weeks intervals. Blood was collected weekly for monitoring antibody titers. After 2 weeks of the third time immunization (3Y), mice were challenged with 10⁵ per mouse 4T1. After tumor challenge, the positive control group, we treat the mice with anti-mouse CTLA-4 antibody (clone 9H10) twice a week for up to three weeks, and the negative control group was treated with PBS. Tumor volume was calculated as: Tumor volume (LWW)=(Length×Width×Width)/2. FIG. 4B shows the mean value of tumor growths in BALB/c mice were vaccinated with MVF-CTLA-4 (59), MVF-CTLA-4 (75), MVF-CTLA-4 (92) or MVF-CTLA-4 (130), PBS as negative control and anti-mouse CTLA-4 antibody (clone 9H10) as positive control. Two-way ANOVA was used to analyze the whole curves of tumor growth, which shows significant difference with p<0.01. FIG. 4C shows individual mouse 4T1 tumor growths in each group of mice were vaccinated with MVF-CTLA-4 (59), MVF-CTLA-4 (75), MVF-CTLA-4 (92) or MVF-CTLA-4 (130), PBS as negative control and anti-mouse CTLA-4 antibody (clone 9H10) as positive control. FIG. 4D shows plots of tumor volume LWW at day 14 for each of the four treatment immunized groups together with PBS control and mAb (9H10) groups; one-way ANOVA was used to analysis multiple groups comparison, which indicated both of p<0.01. Between groups versus with PBS group, mice in CTLA-4 (59), CTLA-4 (75) and CTLA-4 (130) group with significantly smaller tumor size versus with PBS group. Plots of tumor volume LWW at day 16 for each of the four treatment immunized groups together with PBS control and mAb (9H10) groups; one-way ANOVA was used to analysis multiple groups comparison, which indicated both of p<0.01. Between groups versus with PBS group, mice in CTLA-4 (59), CTLA-4 (75) and CTLA-4 (92) group with significantly smaller tumor size versus with PBS group. ** indicates p<0.01, * indicates p<0.05, ns indicates no significant difference. FIG. 4E shows 6-8 weeks old BALB/c mice were vaccinated with MVF-CTLA-4 (59), MVF-CTLA-4 (75), MVF-CTLA-4 (92) or MVF-CTLA-4 (130) for 3 weeks interval with two boost. Mice were challenged with 10⁵ per mouse 4T1 tumor cells. After tumor challenge, the positive control group, we treat the mice with anti-mouse CTLA-4 antibody (clone 9H10) twice a week for up to three weeks, and the negative control group was treated with PBS. The survival curves Log-rank test for multiple groups p<0.01. Between groups comparison of each individual group versus PBS group p<0.05 as indicated with *. * indicates p<0.05; ns indicates no significant difference;

FIGS. 5A-5E show CTLA-4 Peptide Vaccines in D2F2-BALB/c Tumor Model. FIG. 5A shows 6-8 weeks old BALB/c mice were vaccinated with MVF-CTLA-4 (59), MVF-CTLA-4 (75), MVF-CTLA-4 (92) or MVF-CTLA-4 (130) for 3 weeks interval. 0.1 mg peptide cancer vaccine mixed with ISA 720 (1:1) used per mouse. Mice were boosted with the designed doses for every 3 weeks intervals. Blood was collected weekly for monitoring antibody titers. After 2 weeks of the third time immunization (3Y), mice were challenged with 2×10⁵ per mouse D2F2 mammary tumor cells. After tumor challenge, the positive control group, we treat the mice with anti-mouse CTLA-4 antibody (clone 9H10) twice a week for up to three weeks, and the negative control group was treated with PBS. Tumor volume was calculated as: Tumor volume (LWW)=(Length×Width×Width)/2. FIG. 5B shows the mean value of tumor growths in BALB/c mice were vaccinated with MVF-CTLA-4 (59), MVF-CTLA-4 (75), MVF-CTLA-4 (92) or MVF-CTLA-4 (130), PBS as negative control and anti-mouse CTLA-4 antibody (clone 9H10) as positive control. Two-way ANOVA was used to analyze the whole curves of tumor growth, which shows significant difference with p<0.01. FIG. 5C shows Individual mouse D2F2 tumor growths in each group of mice were vaccinated with MVF-CTLA-4 (59), MVF-CTLA-4 (75), MVF-CTLA-4 (92) or MVF-CTLA-4 (130), PBS as negative control and anti-mouse CTLA-4 antibody (clone 9H10) as positive control. FIG. 5D shows plots of tumor volume LWW at day 14 for each of the four treatment immunized groups together with PBS control and mAb (9H10) groups; one-way ANOVA was used to analysis multiple groups comparison, which indicated both of p<0.01. Between groups versus with PBS group, mice in mAb (9H10), CTLA-4 (59), CTLA-4 (75) and CTLA-4 (130) group with significantly smaller tumor size versus with PBS group. Plots of tumor volume LWW at day 16 for each of the four treatment immunized groups together with PBS control and mAb (9H10) groups; one-way ANOVA was used to analysis multiple groups comparison, which indicated both of p<0.01. Between groups versus with PBS group, mice in mAb (9H10), CTLA-4 (59), CTLA-4 (75), CTLA-4 (92) and CTLA-4 (130) group with significantly smaller tumor size versus with PBS group. Plots of tumor volume LWW at day 19 for each of the four treatment immunized groups together with PBS control and mAb (9H10) groups; one-way ANOVA was used to analysis multiple groups comparison, which indicated both of p<0.01. Between groups versus with PBS group, mice in mAb (9H10), CTLA-4 (59), CTLA-4 (75), CTLA-4 (92) and CTLA-4 (130) group with significantly smaller tumor size versus with PBS group. Plots of tumor volume LWW at day 21 for each of the four treatment immunized groups together with PBS control and mAb (9H10) groups; one-way ANOVA was used to analysis multiple groups comparison, which indicated both of p<0.01. Between groups versus with PBS group, mice in mAb (9H10), CTLA-4 (59), CTLA-4 (75), CTLA-4 (92) and CTLA-4 (130) group with significantly smaller tumor size versus with PBS group. ** indicates p<0.01, * indicates p<0.05, ns indicates no significant difference. FIG. 5E shows 6-8 weeks old BALB/c mice were vaccinated with MVF-CTLA-4 (59), MVF-CTLA-4 (75), MVF-CTLA-4 (92) or MVF-CTLA-4 (130) for 3 weeks interval with two boost. Mice were challenged with 2×10⁵ per mouse D2F2 tumor cells. After tumor challenge, the positive control group, we treat the mice with anti-mouse CTLA-4 antibody (clone 9H10) twice a week for up to three weeks, and the negative control group was treated with PBS. The survival curves Log-rank test for multiple groups p<0.01. Between groups comparison of each individual group versus PBS group p<0.05 as indicated. ** indicates p<0.01, * indicates p<0.05; ns indicates no significant difference;

FIGS. 6A, 6B, 6C, and 6D show the CTLA-4 Peptide Mimics in CT26-BALB/c Tumor Model (V32). FIG. 6A shows a schematic of CTLA-4 peptide epitopes therapeutic experiment after mice were challenged with CT26 tumor cells. 6-8 weeks old BALB/c mice were challenged with 1×10⁵ per mouse CT26 tumor cells. After tumor challenge, the positive control group, we treat the mice with anti-mouse CTLA-4 antibody (clone 9H10) twice a week, start from day 1 post tumor challenge and the negative control group was treated with PBS. For the CTLA-4 peptide mimic treatment groups, mice were treated with 200 ug peptide mimics as indicated start from day 1 post tumor challenging. All the mice had been treated at day 1, day 2, day 5, day 7, day 9, day 12, day 14, and day 16 post tumor challenge. The immunogenicity (antibody titer) was under detectable. Tumor volume was calculated as: Tumor volume (LWW)=(Length×Width×Width)/2. FIG. 6B shows the mean value of tumor growths in BALB/c mice treated with CTLA-4 peptide mimics or anti-mouse CTLA-4 antibody as positive control, PBS as negative control. Two-way ANOVA was used to analyze the whole curves of tumor growth, which shows significant difference with p<0.01. FIG. 6C shows tumor volume comparisons. Due to 6/10 PBS mice were removed on Day 14, two more mice were removed on Day 15, all the mice in PBS group were gone on Day 16, so no further tumor volume comparisons after Day 14. At Day 9: one-way ANOVA p<0.01. Between groups comparisons all the treatment groups vs PBS with significant difference. At Day 12: one-way ANOVA p<0.01. Between groups comparisons only 9H10 vs PBS with p<0.01, all the other treatment groups without statistics difference compared with PBS. At Day 14: one-way ANOVA p<0.01. Between groups comparisons: 9H10 vs PBS with p<0.01, CTLA-4 \textcircled{9} RIL (150-130) vs PBS p<0.05. All the left treatment groups without statistics difference compared with PBS. FIG. 6D shows survival curves comparison Log-rank (Mantel-Cox) test. Between groups comparisons all the treatment groups vs PBS with significant difference. * indicates p<0.05; ** indicates p<0.01.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

The term “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. Embodiments defined by each of these transition terms are within the scope of this invention.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

The terms “treat”, “treating”, “treatment” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, pallatively or remedially. In some instances, the terms “treat”, “treating”, “treatment” and grammatical variations thereof, include partially or completely reducing the size of a tumor, reducing the number of tumors, and reducing the severity/metastatic ability of a tumor as compared with prior to treatment of the subject or as compared with the incidence of such symptom in a general or study population.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound. As used herein, a “wt. %” or “weight percent” or “percent by weight” of a component, unless specifically stated to the contrary, refers to the ratio of the weight of the component to the total weight of the composition in which the component is included, expressed as a percentage.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular CTLA-4 peptide (including, but not limited to chimeric or synthetic CTLA-4 peptides) is disclosed and discussed and a number of modifications that can be made to a number of molecules including the CTLA-4 peptide (including, but not limited to chimeric or synthetic CTLA-4 peptides) are discussed, specifically contemplated is each and every combination and permutation of the CTLA-4 peptide (including, but not limited to chimeric or synthetic CTLA-4 peptides) and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Cytotoxic T-lymphocyte-associated protein-4 (CTLA-4; CD152) is one of the inhibitory immune checkpoints expressed on activated T cells and Treg cells. CTLA-4, as a type 1 transmembrane glycoprotein, belongs to the immunoglobulin superfamily. Its gene is located on band q33 of chromosome 2 and encodes for a protein of 223 amino acids. CTLA-4 is a member of CD28-B7 immunoglobulin superfamily of immune regulatory molecules which acts as a negative regulator of T cell activation, especially CD28-dependent T cell responses. The ligands for CTLA-4 are the B7 family members B7-1 (CD80) and B7-2 (CD86). Signaling through the immune checkpoint CTLA-4 enables tumor progression by dampening antitumor immune responses. Therapeutic blockade of the signaling axis between CTLA-4 and its ligands B7-1/B7-2 with monoclonal antibodies has shown remarkable clinical success in the treatment of cancer and demonstrated impressive activity across a broad set of cancer subtypes. Disclosed herein, are improvements on traditional CTLA-4 blockades using smaller, non-antibody peptide therapeutics and peptide vaccines which directly block the interaction of CTLA-4 and B7-1/B7-2 or can stimulate host immune responses to generate antibodies to CTLA-4 that block the B7-1/B7-2 interaction.

Using computer aided analysis of CTLA-4 B cell epitopes, sequences corresponding to CTLA-4 (SEQ ID NO: 1) residues 59-77, 75-92, 92-114, and 130-150 were derived. Thus, in one aspect, disclosed herein are synthetic CTLA-4 peptides for stimulating an immune response to a CTLA-4 protein comprising residues 59-77, 75-92, 92-114, and/or 130-150 of CTLA-4. For example, disclosed herein are synthetic CTLA-4 peptides for stimulating an immune response to a CTLA-4 protein comprising EYASPGKATEVRVTVLRQA (SEQ ID NO: 2) (CTLA-4 residues 59-77), RQADSQVTEVCAATYMMG (SEQ ID NO: 3) (CTLA-4 residues 75-92), GNELTFLDDSICTGTSSGNQVNFHMSVVRARRNDSGTYL (SEQ ID NO: 4) (CTLA-4 residues 92-114), and/or KVELMYPPPYYLGIGNGTQIY (SEQ ID NO: 5) (CTLA-4 residues 130-150). In one aspect, the peptides can acylated and/or amidated. Thus, disclosed herein are synthetic CTLA-4 peptides for stimulating an immune response to a CTLA-4 protein comprising (SEQ ID NO: 2), (SEQ ID NO: 3), (SEQ ID NO: 4), and/or (SEQ ID NO: 5); wherein the synthetic peptide is acylated and/or amidated as set forth in SEQ ID NO: 27, SEQ ID NO: 28, SEQ ID NO: 29, and/or SEQ ID NO: 30.

In some instances uses of an analog of the L-amino sequence can advantages to the base sequence such as resistance to degradation, stability, ease of synthesis, or have greater efficacy. In one aspect, it is understood and herein contemplated that the disclosed synthetic sequences can comprise the L-amino sequence in reverse order from amino to carboxy end. For example, the retro sequence of SEQ ID NO: 2, SEQ ID NO: 3, SEQ ID NO: 4, and SEQ ID NO: 5, are AQRLVTVRVETAKGPSAYE (SEQ ID NO: 12), GMMYTAACVETVQSDAQR (SEQ ID NO: 13), LYTGSDNRRARVVSMHFNVQNGSSTGTCISDDLFTLENG (SEQ ID NO: 14), and YIQTGNGIGLYYPPPYMLEVK (SEQ ID NO: 15), respectively. These retro sequences can also have the mirror conformation of the base sequence. Thus, disclosed herein are synthetic CTLA-4 peptides comprising one or more of the sequences as set forth in SEQ ID NO: 12, SEQ ID NO: 13, SEQ ID NO: 14, and/or SEQ ID NO: 15. As with SEQ ID NO: 2, SEQ ID NO: 3, SEQ ID NO: 4, and SEQ ID NO: 5; synthetic peptides comprising SEQ ID NO 12, SEQ ID NO: 13, SEQ ID NO: 14 and/or SEQ ID NO: 15 can be acetylated and/or amidated as set forth in SEQ ID NO: 31, SEQ ID NO: 32, SEQ ID NO: 33, and/or SEQ ID NO: 34, respectively.

In addition to retro analogs of the L-amino acid sequence set forth in SEQ ID NO: 2, SEQ ID NO: 3, SEQ ID NO: 4, and SEQ ID NO: 5 which are set forth in SEQ ID NO 12, SEQ ID NO: 13, SEQ ID NO: 14 and SEQ ID NO: 15 are D enantiomer analogs of the forward L-amino (SEQ ID NO: 2, SEQ ID NO: 3, SEQ ID NO: 4, and SEQ ID NO: 5) and retro L-amino sequence (SEQ ID NO 12, SEQ ID NO: 13, SEQ ID NO: 14 and SEQ ID NO: 15) which can possess increased resistance to degradation and proteolysis allowing for better oral administration, extended efficacy, and increased ease of synthesis. Accordingly, in one aspect, disclosed herein are synthetic CTLA-4 peptide (including, but not limited to chimeric or synthetic CTLA-4 peptides) peptides comprising one or more of SEQ ID NO: 2, SEQ ID NO: 3, SEQ ID NO: 4, SEQ ID NO: 5, SEQ ID NO 12, SEQ ID NO: 13, SEQ ID NO: 14 and/or SEQ ID NO: 15; wherein the amino acids comprising the sequence are D amino acids as set forth in SEQ ID NO: 35, SEQ ID NO: 36, SEQ ID NO: 37, SEQ ID NO: 38, SEQ ID NO: 47, SEQ ID NO: 48, SEQ ID NO: 49, and DEQ ID NO: 50. As with the L amino acids, the D enantiomers SEQ ID NO: 35, SEQ ID NO: 36, SEQ ID NO: 37, SEQ ID NO: 38, SEQ ID NO: 47, SEQ ID NO: 48, SEQ ID NO: 49, and DEQ ID NO: 50 can be acetylated and/or amidated as set forth in SEQ ID NO: 39, SEQ ID NO: 40, SEQ ID NO: 41, SEQ ID NO: 42, SEQ ID NO: 51, SEQ ID NO: 52, SEQ ID NO: 53, and DEQ ID NO: 54.

In one aspect, it is understood and herein contemplated that the disclosed synthetic CTLA-4 peptides can have increased B cell stimulation by linking the synthetic CTLA-4 peptides to a helper T (Th) cell epitope that promotes the release of cytokines that assist in bypassing MHC restriction (i.e., a promiscuous Th cell epitope) to form a chimeric CTLA-4 peptide. For example, disclosed herein, in one aspect are CTLA-4 chimeric peptides for stimulating an immune response to a CTLA-4 protein comprising one or more CTLA-4 B cell epitopes further comprising a T helper (Th) epitope (for example, a measles virus fusion protein peptide such as SEQ ID NO: 6), wherein the one or more CTLA-4 B cell epitopes consist of a sequence selected from the group consisting of the SEQ ID NO: 2, SEQ ID NO: 3, SEQ ID NO: 4, SEQ ID NO: 5, SEQ ID NO: 12, SEQ ID NO: 13, SEQ ID NO: 14, and/or SEQ ID NO: 15; the resulting sequence being SEQ ID NO: 8, SEQ ID NO: 9, SEQ ID NO: 10, SEQ ID NO: 11, SEQ ID NO: 16, SEQ ID NO: 17, SEQ ID NO: 18, and/or SEQ ID NO: 19, respectively. It is understood and herein contemplated that the B cell epitope (i.e., the CTLA-4 synthetic peptide) can also comprise D enantiomer amino acids of SEQ ID NO: 8, SEQ ID NO: 9, SEQ ID NO: 10, SEQ ID NO: 11, SEQ ID NO: 16, SEQ ID NO: 17, SEQ ID NO: 18, and/or SEQ ID NO: 19 as set forth in SEQ ID NO: 43, SEQ ID NO: 44, SEQ ID NO: 45, SEQ ID NO: 46, SEQ ID NO: 55, SEQ ID NO: 56, SEQ ID NO: 57, and/or SEQ ID NO: 58, respectively.

The Th epitope can be from about 14 to about 22, more preferably, about 15 to 21, most preferably 16 amino acids in length. Preferably, the Th cell epitope has one of the following amino acid sequences provided in Table 1.

TABLE 1

| Peptide | | SEQ |
|---|---|---|
| Designation | Sequence | ID NO: |
| MVF | KLLSLIKGVIVHRLEGVE | 6 |
| TT | NSVDDALINSTIYSYFPSV | 20 |
| TT1 | PGINGKAIHLVNNQSSE | 21 |
| P2 | QYIKANSKFIGITEL | 22 |
| P30 | FNNFTVSFWLRVPKVSASHLE | 23 |
| MVF (natural) | LSEIKGVIVHRLEGV | 24 |
| HBV | FFLLTRILTIPQSLN | 25 |
| CSP | TCGVGVRVRSRVNAANKKPE | 26 |

To join the synthetic CTLA-4 peptide and the Th cell epitope, an amino acid linker can be used. Preferably the linker is a peptide of from about 2 to about 15 amino acids, more preferably from about 2 to about 10 amino acids, most preferably from about 2 to about 6 amino acids in length. The most preferred linker comprises the amino acid sequence Gly-Pro-Ser-Leu (SEQ ID NO: 7). Thus, in one aspect, also disclosed herein are chimeric peptides comprising the synthetic peptide of any preceding aspect, further comprising a Th epitope (for example, a measles virus fusion protein peptide such as SEQ ID NO: 6), and a linker (such as, for example, SEQ ID NO: 7) joining the synthetic CTLA-4 peptide to the Th epitope. For example, disclosed herein, in one aspect, are chimeric CTLA-4 peptides for stimulating an immune response to a CTLA-4 protein comprising one or more CTLA-4 B cell epitopes, a T helper (Th) epitope (for example, a measles virus fusion protein peptide such as SEQ ID NO: 6), and a linker (such as, for example, SEQ ID NO: 7) joining the CTLA-4 B cell epitope to the Th epitope; wherein the chimeric CTLA-4 peptide comprises the amino acid sequence as set forth in

(SEQ ID NO: 8)
KLLSLIKGVIVHRLEGVEGPSLEYASPGKATEVRVTVLRQA,
(SEQ ID NO: 9)
KLLSLIKGVIVHRLEGVEGPSLRQADSQVTEVCAATYMMG,
(SEQ ID NO: 10)
KLLSLIKGVIVHRLEGVEGPSLGNELTFLDDSICTGTSSGNQVNFHMSV
VRARRNDSGTYL,
(SEQ ID NO: 11)
KLLSLIKGVIVHRLEGVEGPSLKVELMYPPPYYLGIGNGTQIY,
(SEQ ID NO: 16)
KLLSLIKGVIVHRLEGVEGPSLAQRLVTVRVETAKGPSAYE,
(SEQ ID NO: 17)
KLLSLIKGVIVHRLEGVEGPSLGMMYTAACVETVQSDAQR,
(SEQ ID NO: 18)
KLLSLIKGVIVHRLEGVEGPSLLYTGSDNRRARVVSMHFNVQNGSSTGT
CISDDLFTLENG,
and/or
(SEQ ID NO: 19)
KLLSLIKGVIVHRLEGVEGPSLYIQTGNGIGLYYPPPYMLEVK.

As with the synthetic peptides, it is understood and herein contemplated that the amino acids of the synthetic CTLA-4 peptides comprised within the chimeric CTLA-4 peptides can be a D amino acid analogs of the L-amino acids in the sequence. Accordingly, in one aspect, disclosed herein are chimeric peptides comprising any of the synthetic CTLA-4 peptides disclosed herein, further comprising a Th epitope (for example, a measles virus fusion protein peptide such as SEQ ID NO: 6), and a linker (such as, for example, SEQ ID NO: 7) joining the synthetic CTLA-4 peptide to the Th epitope. For example, disclosed herein, in one aspect, are chimeric CTLA-4 peptides comprising the amino acid sequence as set forth in SEQ ID NO: 35, SEQ ID NO: 36, SEQ ID NO: 37, SEQ ID NO: 38, SEQ ID NO: 47, SEQ ID NO: 48, SEQ ID NO: 49, and/or SEQ ID NO: 50; wherein the synthetic CTLA-4 peptide sequence (i.e., the B cell epitope) comprises D amino acids. In such instances, the Th epitope and the liner are L-amino acids while the B cell epitope comprises D-amino acids. For example, residues 1-22 of SEQ ID NO: 43 and SEQ ID NO: 55 are L-amino acids while residues 23-41 are D-amino acids. Similarly, residues 1-22 of SEQ ID NO: 44 and SEQ ID NO: 56 are L-amino acids while residues 23-40 are D-amino acids; residues 1-22 of SEQ ID NO: 45 and SEQ ID NO: 57 are L-amino acids while residues 23-61 are D-amino acids; and residues 1-22 of SEQ ID NO: 46 and SEQ ID NO: 58 are L-amino acids while residues 23-43 are D-amino acids.

### 1. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2:482 (1981), by the homology alignment algorithm of Needleman and Wunsch, J. Mol Biol. 48:443 (1970), by the search for similarity method of Pearson and Lipman, Proc. Natl. Acad. Sci. U.S.A. 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

### 2. Peptides

### a) Protein and Peptide Variants

As discussed herein there are numerous variants of the synthetic CTLA-4 peptides and chimeric CTLA-4 peptides that are known and herein contemplated. In addition, to the known functional CTLA-4 strain variants there are derivatives of the synthetic CTLA-4 peptides and chimeric CTLA-4 peptides which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 2 and 3 and are referred to as conservative substitutions.

TABLE 2
Amino Acid Abbreviations

| | |
|---|---|
| Amino | |
| Acid | Abbreviations |

| | | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 3
Amino Acid Substitutions
Original Residue Exemplary Conservative Substitutions,
others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 3, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted 10 for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90% or 95% identity to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2:482 (1981), by the homology alignment algorithm of Needleman and Wunsch, J. Mol. Biol. 48:443 (1970), by the search for similarity method of Pearson and Lipman, Proc. Natl. Acad. Sci. U.S.A. 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that peptide or protein is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 2 and Table 3. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way.

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. 1 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. In other words, contemplated herein is the inverso (i.e., the D-amino acid substitution) of any disclosed sequence. Cysteine residues can be used to cyclize or attach two or more peptides together. This can be beneficial to constrain peptides into particular conformations. In one aspect, disclosed herein are synthetic CTLA-4 peptides comprising one or more of the sequences as set forth in SEQ ID NO:2, SEQ ID NO:3, SEQ ID NO:4, or SEQ ID NO:5; wherein the amino acids of the peptide are the D enantiomer.

In one aspect, the disclosed synthetic peptides can be in reverse order such that the amino to carboxy end of the peptide is reversed (i.e., the retro sequence). In one aspect, disclosed herein are the retro sequences of SEQ ID NO: 2, SEQ ID NO: 3, SEQ ID NO: 4, and SEQ ID NO: 5, which comprises, SEQ ID NO: 12, SEQ ID NO: 13, SEQ ID NO: 14, and SEQ ID NO: 15, respectively. These retro sequences can also have the mirror conformation of the base sequence. In one aspect, the retro sequence can also comprise a D amino acid substitution (i.e., the retro-inverso) sequence. Thus, disclosed herein are synthetic CTLA-4 peptides comprising one or more of the sequences as set forth in SEQ ID NO: 12, SEQ ID NO: 13, SEQ ID NO: 14, and SEQ ID NO: 15; wherein the amino acids of the peptide are the D enantiomer as set forth in SEQ ID NO: 47, SEQ ID NO: 48, SEQ ID NO: 49, and SEQ ID NO: 50, respectively.

It is understood that any of the D amino acid substituted synthetic peptides disclosed herein can be used as the CTLA-4 epitope in the disclosed CTLA-4 chimeric peptides. For example, disclosed herein are chimeric CTLA-4 peptides comprising one or more CTLA-4 B cell epitopes, a T helper (Th) epitope (for example, a measles virus fusion protein peptide such as SEQ ID NO: 6), and a linker joining the CTLA-4 B cell epitope to the Th epitope (such as, for example SEQ ID NO: 7), wherein the one or more CTLA-4 B cell epitopes consist of a sequence selected from the group consisting of SEQ ID NO: 2, SEQ ID NO: 3, SEQ ID NO: 4, SEQ ID NO: 12, SEQ ID NO: 13, SEQ ID NO: 14, and SEQ ID NO: 15; and wherein the amino acids of the peptide are the D enantiomer. In one aspect, disclosed herein are chimeric CTLA-4 peptides, wherein the peptide comprises the amino acid sequence as set forth in SEQ ID NO: 8, SEQI DNO: 9, SEQ ID NO: 10 SEQ ID NO: 11, SEQ ID NO: 16, SEQ ID NO: 17, SEQ ID NO: 18, or SEQ ID NO: 19; and wherein the amino acids of the synthetic CTLA-4 peptide are the D enantiomer as set forth in SEQ ID NO: 43, SEQ ID NO: 44, SEQ ID NO: 45, SEQ ID NO: 46, SEQ ID NO: 55, SEQ ID NO: 56, SEQ ID NO: 57, and SEQ ID NO: 58, respectively.

### 3. Pharmaceutical Carriers/Delivery of Pharmaceutical Products

As described above, the synthetic CTLA-4 peptides and chimeric CTLA-4 peptides disclosed herein can also be administered in vivo in a pharmaceutically acceptable carrier. Thus, in one aspect, disclosed herein are pharmaceutical composition comprising any one or more of the CTLA-4 peptides as set forth in SEQ ID NO: 2, SEQ ID NO: 3, SEQ ID NO: 4, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, and/or SEQ ID NO: 58.

By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

It is understood and herein contemplated that the disclosed CTLA-4 peptides comprising pharmaceutical compositions are particularly useful in the treatment of diseases or conditions where CTLA-4 mediated immune suppression occurs. Thus, in one aspect, the disclosed pharmaceutical composition comprising one or more of the CTLA-4 peptides disclosed herein can be combined with a disease-specific treatment or vaccine to further increase the efficacy of the CTLA-4 peptides. Accordingly, in one aspect, disclosed herein are pharmaceutical compositions comprising one or more of the CTLA-4 peptide, synthetic peptides, or chimeric peptides disclosed herein (for example, SEQ ID NO: 2, SEQ ID NO: 3, SEQ ID NO: 4, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, and/or SEQ ID NO: 58) further comprising a disease-specific treatment or vaccine.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and Mckenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

The synthetic CTLA-4 peptides, chimeras, and antibodies disclosed herein that inhibit the interaction of CTLA-4 and PD-L1 can be administered prophylactically to patients or subjects who are at risk for developing a cancer, autoimmune disease, of Alzheimer's disease or therapeutically (i.e., after diagnosis of a disease or onset of symptoms) for treatment of a cancer, autoimmune disease, of Alzheimer's disease.

Other molecules or antibodies that interact with CTLA-4 or B7-1/B7-2 to inhibit CTLA-4/B7-1/B7-2 interactions (for example, ipilimumab and tremelimumab) can be used in combination with the disclosed CTLA-4 peptides, synthetic CTLA-4 peptides, chimeric CTLA-4 peptides, or anti-CTLA-4 antibodies to treat a cancer. Other molecules or antibodies that act agonistically on CTLA-4 (for example abatacept and belatacept) can be used in combination with the disclosed CTLA-4 peptides, synthetic CTLA-4 peptides, chimeric CTLA-4 peptides, or anti-CTLA-4 antibodies to treat, autoimmune disease or Alzheimer's disease in a subject.

### 4. Antibodies

### (1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with CTLA-4 such that CTLA-4 is inhibited from interacting with B7-1/B7-2. Antibodies that bind SEQ ID NO: 1, SEQ ID NO: 2, SEQ ID NO: 3, SEQ ID NO: 4, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, and/or SEQ ID NO: 58 involved in the interaction between CTLA-4 and B7-1/B7-2 are also disclosed. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively. 92. The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or fragments thereof” encompasses chimeric antibodies and hybrid antibodies, with dual or multiple antigen or epitope specificities, and fragments, such as F (ab′) 2, Fab′, Fab, Fv, sFv, and the like, including hybrid fragments. Thus, fragments of the antibodies that retain the ability to bind their specific antigens are provided. For example, fragments of antibodies which maintain CTLA-4 binding activity or bind SEQ ID NO: 1, SEQ ID NO: 2, SEQ ID NO: 3, SEQ ID NO: 4, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, and/or SEQ ID NO: 58 are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

### (2) Human Antibodies

The disclosed human antibodies can be prepared using any technique. The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J (H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

### (3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′)2, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody.

### (4) Administration of Antibodies

Administration of the antibodies can be done as disclosed herein. Nucleic acid approaches for antibody delivery also exist. The broadly neutralizing anti-CTLA-4 antibodies and antibody fragments (including any antibody that binds to SEQ ID NO: 1, SEQ ID NO: 2, SEQ ID NO: 3, SEQ ID NO: 4, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, and/or SEQ ID NO: 58) can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

### C. METHOD OF TREATING DISEASE

It is understood and herein contemplated that the disclosed compositions, synthetic CTLA-4 peptides, and chimeric CTLA-4 peptides can be used to treat any disease where immune suppression and prevention of programmed cell death is advantageous to the disease, such as Alzheimer's disease, autoimmune diseases, or any disease where uncontrolled cellular proliferation occurs such as cancers.

A non-limiting list of different types of autoimmune disease that can be treated using the chimeric or synthetic peptides or pharmaceutical compositions disclosed herein includes, but is not limited to, Psoriasis, Alopecia Areata, Primary biliary cirrhosis, Autoimmune polyendocrine syndrome, Diabetes mellitus type 1, autoimmune thyroiditis, Systemic Lupus Erythematosus, Multiple sclerosis, Guillain-Barré syndrome, Grave's disease, Sjogren's syndrome, ulcerative colitis, Autoimmune hemolytic anemia, Pernicious anemia, Psoriatic arthritis, rheumatoid arthritis, relapsing polychondritis, myasthenia gravis, Acute disseminated encephalomyelitis, and Granulomatosis with polyangiitis.

A non-limiting list of different types of cancers that can be treated using the chimeric or synthetic peptides or pharmaceutical compositions disclosed herein includes, but is not limited to, lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumors, myelomas, AIDS-related lymphomas or sarcomas, metastatic cancers, or cancers in general.

A representative but non-limiting list of cancers that the disclosed compositions, chimeric peptides, and synthetic peptides can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, ipilimumab-refractory melanoma, or pancreatic cancer.

Accordingly, in one aspect, disclosed herein are methods of treating a cancer, Alzheimer's disease, or an autoimmune disease in a subject comprising administering to a subject a CTLA-4 synthetic peptide, wherein the CTLA-4 synthetic peptide comprises one or more of the sequences as set forth in SEQ ID NO:2, SEQ ID NO:3, SEQ ID NO:4, SEQ ID NO: 5, SEQ ID NO: 12, SEQ ID NO: 13, SEQ ID NO: 14, or SEQ ID NO: 15. It is understood and herein contemplated that the synthetic peptides can comprise be acetylated, amidated, and/or the D enantiomer as set forth in SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 47, SEQ ID NO: 48, SEQ ID NO: 49, SEQ ID NO: 50, SEQ ID NO: 51, SEQ ID NO: 52, SEQ ID NO: 53, and/or SEQ ID NO: 54. Accordingly, in one aspect, disclosed herein are methods of treating a cancer, Alzheimer's disease, or an autoimmune disease in a subject comprising administering to a subject a CTLA-4 synthetic peptide wherein the CTLA-4 synthetic peptide comprises the D enantiomer and or D enantiomer retro-inverso as set forth in SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 47, SEQ ID NO: 48, SEQ ID NO: 49, SEQ ID NO: 50, SEQ ID NO: 51, SEQ ID NO: 52, SEQ ID NO: 53, and SEQ ID NO: 54.

It is further understood and herein contemplated that the synthetic peptides for use in treating a cancer, autoimmune disease or Alzheimer's disease can be a component of a chimeric peptide. Thus, in one aspect, disclosed herein are methods of treating a cancer, Alzheimer's disease, or an autoimmune disease in a subject comprising administering to a subject a CTLA-4 chimeric peptide wherein the chimeric peptide comprises one or more CTLA-4 B cell epitopes, a T helper (Th) epitope, and a linker joining the CTLA-4 B cell epitope to the Th epitope, wherein the one or more CTLA-4 B cell epitopes consist of a sequence selected from the group consisting of SEQ ID NO:2, SEQ ID NO:3, SEQ ID NO:4, SEQ ID NO:5, SEQ ID NO: 12, SEQ ID NO: 13, SEQ ID NO: 14, and SEQ ID NO: 15 and/or their D-enatiomers as set forth in SEQ ID NO: 35, SEQ ID NO: 36, SEQ ID NO: 37, SEQ ID NO: 38, SEQ ID NO: 47, SEQ ID NO: 48, SEQ ID NO: 49, and SEQ ID NO: 50. It is understood and herein contemplated that the CTLA-4 peptides (i.e., the CTLA-4 B cell epitopes) used in the chimeric peptides can comprise be acetylated, amidated, and/or the D enantiomer. In one aspect, for example, disclosed herein are methods of treating a cancer, Alzheimer's disease, or an autoimmune disease in a subject comprising administering to a subject a CTLA-4 chimeric peptide wherein the chimeric peptide comprises SEQ ID NO: 8, SEQ ID NO: 9, SEQ ID NO: 10, SEQ ID NO: 11, SEQ ID NO: 16, SEQ ID NO: 17, SEQ ID NO: 18, SEQ ID NO: 19, SEQ ID NO: 43, SEQ ID NO: 44, SEQ ID NO: 45, SEQ ID NO: 46, SEQ ID NO: 55, SEQ ID NO: 56, SEQ ID NO: 57, or SEQ ID NO: 58.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Selection, Design, and Modeling of Peptide Epitopes for CTLA-4

The selection of candidate B-cell epitopes expressed on the surface of CTLA-4 was accomplished by an in-house (Peptide Companion™, 5x.com) computer-aided analysis using six correlates of antigenicity reviewed by Kaumaya et al: (a) The profiles of chain flexibility and mobility of individual sequences were calculated according to Karplus and Schultz; (b) Hydropathy profiles were generated over a seven residue span setting and then smoothed with a three-residue span using the scale of Kyte and Doolittle; (c) Hydrophilicity profiles over a six-residue window were generated using the program of Hopp and Woods; (d) Analysis of the exposure of an amino acid residue to water (1.4 A probe) was carried out by the solvent exposure algorithm of Rose et al.; (e) Protrusion indices were calculated by the method of Thornton et al. that predicts portions of proteins that are accessible and protrude into the solvent; (f) The probability that a five-residue sequence is antigenic was determined by the method of Welling et al.; Sequences were given a score of 1 to 6 based on their respective index values and were ranked: the highest ranking sequences had the highest individual score for the analyses examined (6/6), and successive candidates had the next highest score (5/6), etc. The best scoring epitopes were further ranked by correlation with their secondary structural attributes; e.g., an amphiphilic α-helical sequence or a β-turn loop regions are preferred over a random coil fragments. Computer programs by Chou and Fasman and Novotny et al. were used to predict the secondary structure (α-helix, β-strand/sheet, β-turn/loop, random coil) and α-helical amphiphilic moment. Finally, consideration was given to the individual amino acid sequence. Electrostatic ion pairs and helix dipole interaction in helical segment were also considered (e.g., hydrophobic/hydrophilic balance). The sequences receiving the highest scores are displayed in Table 4. Employing this method, four of the twelve highest scoring B-cell epitope sequences of human CTLA-4 were prioritized. Amino acid 59-77; 75-92; 92-114 and 130-150 were chosen for evaluation in combination with information from the crystal structure of CTLA-4: B7-1/B7-2. The structures of human CTLA-4, B7-1, and B7-2 have been determined, but those in turn did not account for significant plasticity within the human CTLA-4 upon complex formation demonstrated only very recently by the structure of the fully human CTLA-4: B7-1/B7-2. Although the above structures provided a complete description of the interaction, the flat surface of the protein-protein interface still complicates drug design efforts in the absence of structural information on the small-molecule inhibitors in complex with CTLA-4 to guide further rational drug development.

TABLE 4
human CTLA-4 predicted B-cell epitopes

| | | Secondary |
|---|---|---|
| Residue | Sequence | Structure |
| 59-77 | EYASPGKATEVRVTVLRQA (SEQ ID NO: 2) | Anti-parallel β- |
| | | sheet/loop |
| 75-92 | RQADSQVTEVCAATYMMG (SEQ ID NO: 3) | |
| 92-114 | GNELTFLDDSICTGTSSGNQVNFHMSVVRARRNDSGTYL | |
| | (SEQ ID NO: 4) | |
| 130-150 | KVELMYPPPYYLGIGNGTQIY (SEQ ID NO: 5) | |

### 2. Example 2: Synthesis, Purification, and Characterization of CTLA-4 Peptides and MVF-CTLA-4 Peptides

Peptide synthesis was performed using 9600 Milligen/Biosearch solid-phase peptide synthesizer (Millipore, Bedford, MA) using Fmoc/Boc chemistry. Clear amide resin (0.50 mmol/gm) (Peptide International, Louisville, KY) and Fmoc protected amino acids (P3BioSystems, Louisville, KY) were used for synthesis of all of the peptides. In the case of the chimeric peptides, the B cell epitopes were colinearly synthesized with the promiscuous Th MVF (residues 288-302) epitope using regioselective side chain protections and a GPSL linker. Some of the B cell epitopes were acetylated using Acetylimidazole (Sigma-Aldrich St. Louis, MO) in DMF. The peptides were reacted overnight then washed with DMF before cleavage. Peptides were cleaved using reagent R (trifluoroacetic acid:TFA:Thiansole:EDT:Anisole, 90:5:3:2) (Sigma-Aldrich, St. Louis, MO). The crude peptides were purified by reverse-phase HPLC in a gradient system using a C-4 vydac column in water/acetonitrile (0.1% trifluoroacetic acid) on a Waters system. At the end of purification, the pure fractions were then analyzed in analytical HPLC, and fractions of interest were pooled together and lyophilized in 10% acetic acid solution. The final purified peptides listed in Table 5 were then identified using mass spectrometry (Campus Chemical Instrumentation Center, The Ohio State University, Columbus, OH).

TABLE 5
Peptide Sequences of CTLA-4

| Peptides | Amino Acid Sequence of CTLA-4 Peptides |
|---|---|
| CTLA-4 (59-77) | H₂N-EYASPGKATEVRVTVLRQA-CONH₂ (SEQ ID NO: 2) |
| AC-CTLA-4 (59-77) | CH₃CONH-EYASPGKATEVRVTVLRQA-CONH₂ (SEQ ID NO: 27) |
| MVF-CTLA-4 (59-77) | KLLSLIKGVIVHRLEGVEGPSLEYASPGKATEVRVTVLRQA- |
| | CONH₂ (SEQ ID NO: 8) |
| CTLA-4 (75-92) | H₂N-ROADSQVTEVCAATYMMG-CONH₂ (SEQ ID NO: 3) |
| AC-CTLA-4 (75-92) | CH₃CONH-RQADSQVTEVCAATYMMG-CONH₂ (SEQ ID NO: 28) |
| MVF-CTLA-4 (75-92) | KLLSLIKGVIVHRLEGVEGPSLRQADSQVTEVCAATYMMG- |
| | CONH₂ (SEQ ID NO: 9) |
| CTLA-4 (92-114) | H₂N-GNELTFLDDSICTGTSSGNQVNFHMSVVRARRNDSGTYL- |
| | CONH₂ (SEQ ID NO: 4) |
| AC-CTLA-4 (92-114) | CH₃CONH- |
| | GNELTFLDDSICTGTSSGNQVNFHMSVVRARRNDSGTYL- |
| | CONH₂ (SEQ ID NO: 29) |
| MVF-CTLA-4 (92-114) | KLLSLIKGVIVHRLEGVEGPSLGNELTFLDDSICTGTSSGNQVNF |
| | HMSVVRARRNDSGTYL-CONH₂ (SEQ ID NO: 10) |
| CTLA-4 (130-150) | H₂N-KVELMYPPPYYLGIGNGTQIY-CONH₂ (SEQ ID NO: 5) |
| AC-CTLA-4 (130-150) | CH₃CONH-KVELMYPPPYYLGIGNGTQIY-CONH₂ (SEQ ID NO: |
| | 30) |
| MVF-CTLA-4 (130-150) | KLLSLIKGVIVHRLEGVE-GPSL-KVELMYPPPYYLGIGNGTQIY- |
| | CONH₂ (SEQ ID NO: 11) |
| CTLA-4-RI (77-59) | H₂N-AQRLVTVRVETAKGPSAYE-CONH₂ (SEQ ID NO: 12) |
| AC-CTLA-4-RI (77-59) | CH₃CONH-AQRLVTVRVETAKGPSAYE-CONH₂ (SEQ ID NO: |
| | 31) |
| CTLA-4-RI (92-75) | H₂N-GMMYTAACVETVQSDAQR-CONH₂ (SEQ ID NO: 13) |
| AC-CTLA-4-RI (92-75) | CH₃CONH-GMMYTAACVETVQSDAQR-CONH₂ (SEQ ID NO: 32) |
| CTLA-4-RI (114-92) | H₂N- |
| | LYTGSDNRRARVVSMHFNVQNGSSTGTCISDDLFTLENG- |
| | CONH₂ (SEQ ID NO: 14) |
| AC-CTLA-4-RI (114-92) | CH₃CONH- |
| | LYTGSDNRRARVVSMHFNVQNGSSTGTCISDDLFTLENG- |
| | CONH₂ (SEQ ID NO: 33) |
| CTLA-4-RI (150-130) | H₂N-YIQTGNGIGLYYPPPYMLEVK-CONH₂ (SEQ ID NO: 15) |
| AC-CTLA-4-RI (150-130) | CH₃CONH-YIQTGNGIGLYYPPPYMLEVK-CONH₂ (SEQ ID NO: 34) |
| MVF-CTLA-4-RI (77-59) | KLLSLIKGVIVHRLEGVEGPSLAQRLVTVRVETAKGPSAY |
| | E (SEQ ID NO: 16) |
| MVF-CTLA-4-RI (92-75) | KLLSLIKGVIVHRLEGVEGPSLGMMYTAACVETVQSDAQ |
| | R (SEQ ID NO: 17) |
| MVF-CTLA-4-RI (114-92) | KLLSLIKGVIVHRLEGVEGPSLLYTGSDNRRARVVSMHFN |
| | VQNGSSTGTCISDDLFTLENG (SEQ ID NO: 18) |
| MVF-CTLA-4-RI (150-130) | KLLSLIKGVIVHRLEGVEGPSLYIQTGNGIGLYYPPPYMLEVK |
| | (SEQ ID NO: 19) |

### 3. Example 3: CTLA-4 Peptides Selection. SEE CTLA-4 Peptide Binder Prediction, Synthesis, HPLC and Mass Spec Characterization

The secondary structure of peptides plays a pivotal role to the epitopes' crystal structures complex. Four B-cell epitopes of human CTLA-4 were chosen and synthesized for further investigation; they are amino acid 59-77, 75-92, 92-114, and 130-150. We used PyMOL 3-D modeling software to model the secondary structure of peptides' sequences. The published results indicated the CTLA-4/CD28 interaction complex.

### a) Immunogenicity and Antigenicity of Four Novel Human CTLA-4 Peptide Epitopes in NewZeland Rabbits

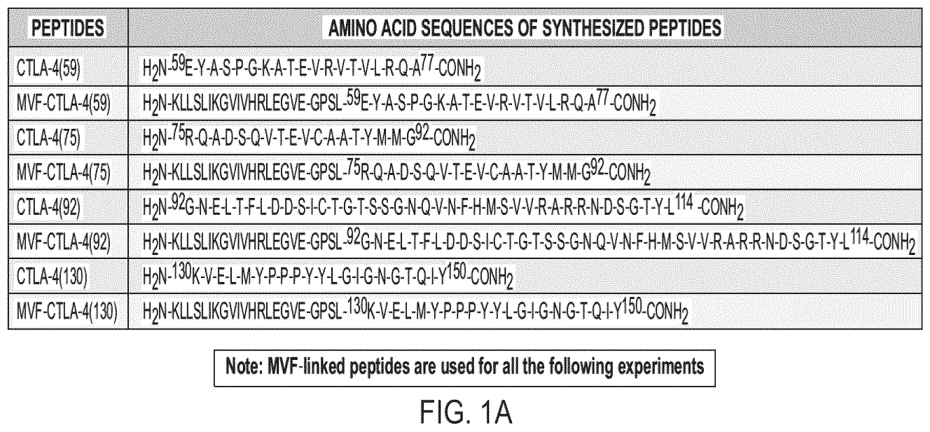

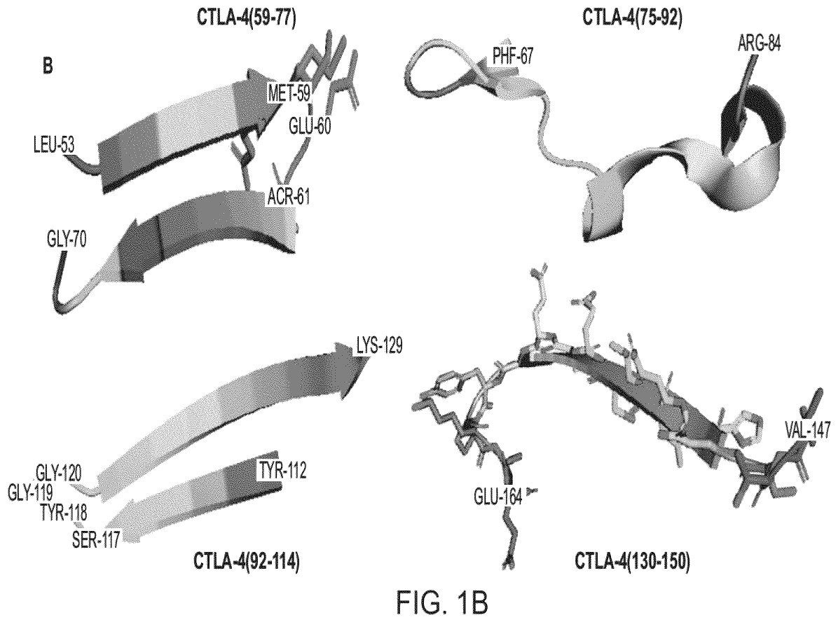

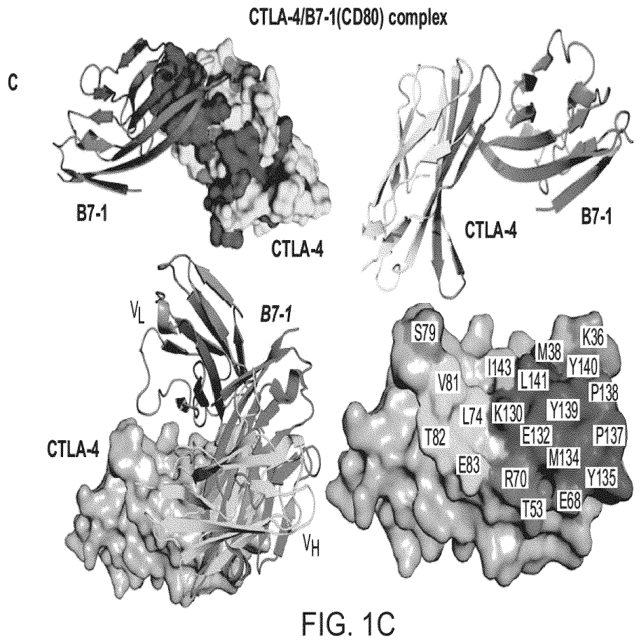

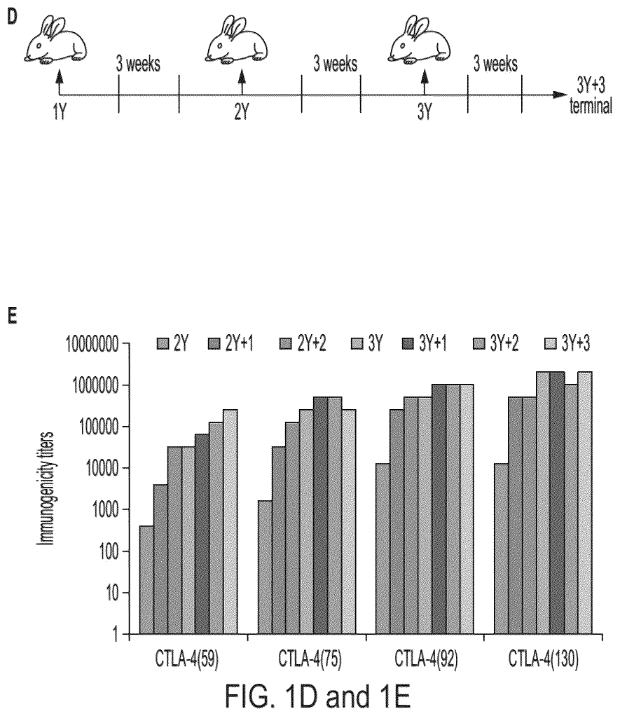

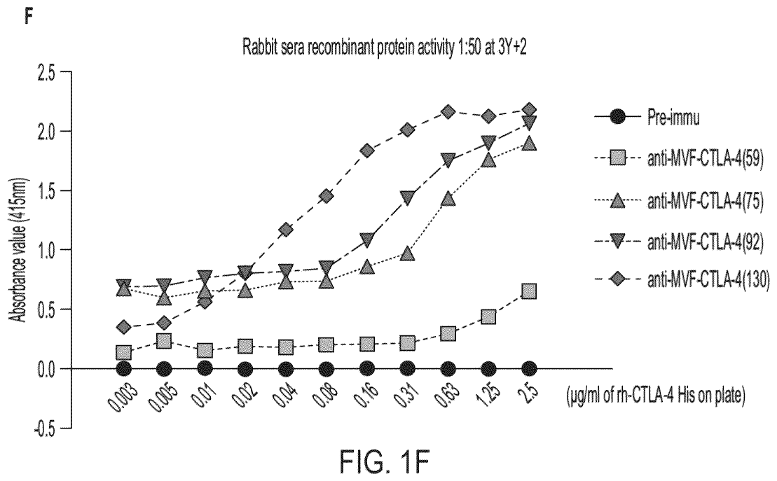

10-week old (2 kg) New Zealand white rabbits were immunized with each of four MVF-CTLA-4 epitope peptide as indicated. After the primary immunization, the rabbits received another two boosts with three weeks intervals (FIG. 1A). The bleed samples were collected weekly after the first boost until the animals were terminated at the three week after the last boost. ELISAs were performed to monitor the immunogenicity of each CTLA-4 epitopes over the experimental period. The antibodies titers are all over 1 to 100 thousands in the terminal bleeds. Which indicate the immunized rabbits generated high titers of antibodies against the immunized peptides (FIG. 1B). Moreover, the antibodies showed relatively high antigenicity against recombinant human CTLA-4, especially anti-MVF-CTLA-4 (130). While, the anti-MVF-CTLA-4 (59) showed relatively lower antigenicity against rh-CTLA-4 compared with other three antibodies (FIG. 1C).

### b) Immunogenicity, Antibodies Isotype Distribution in Immunized BALB/c Mice and Challenged with CT26 Colon Carcinoma, 4T1 and D2F2 Mammary Cancer Cell Lines

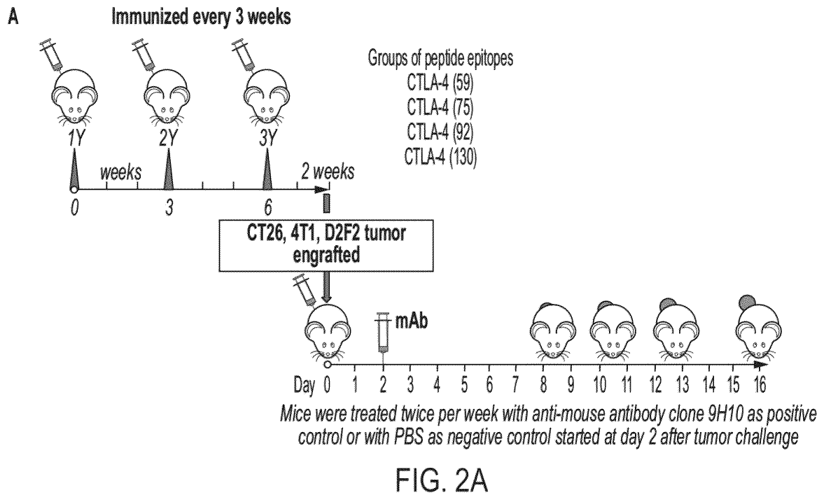

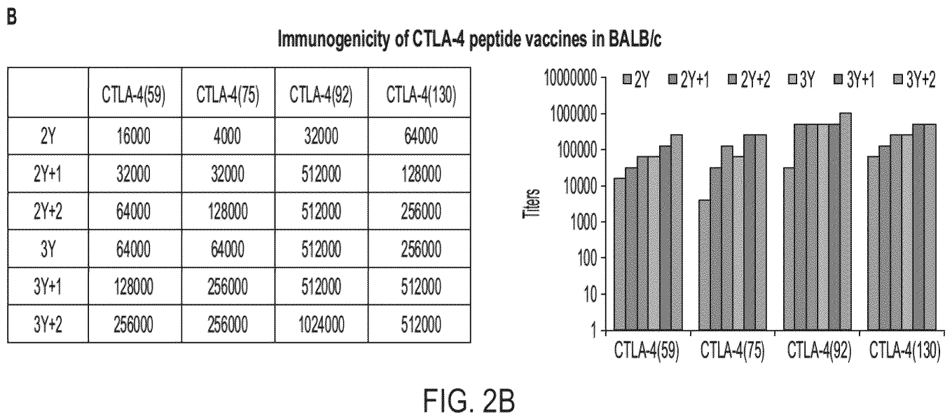

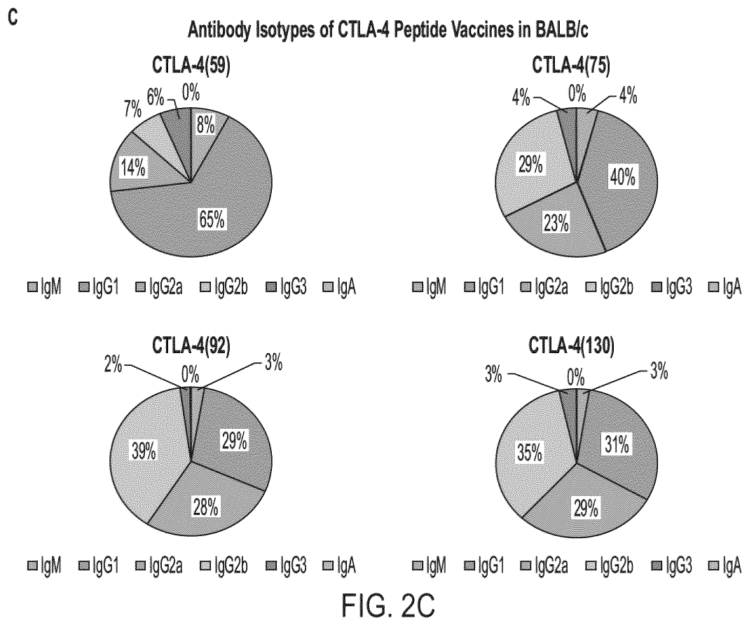

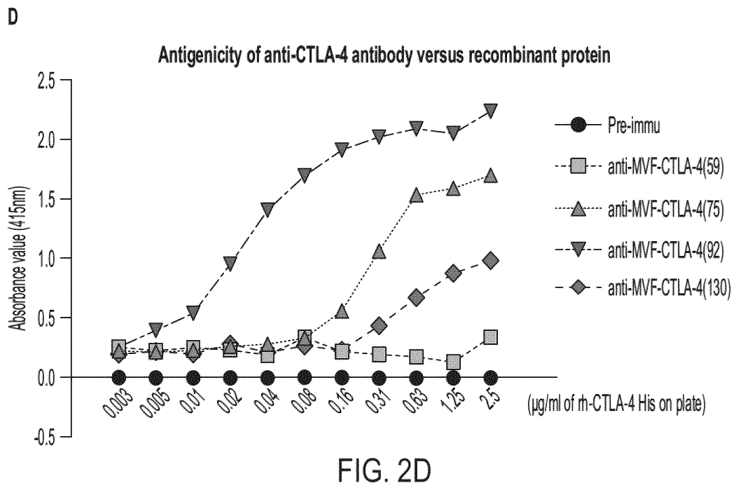

Female BALB/c mice (6-8 weeks old) were immunized with each MVF-CTLA-4 peptide with three weeks intervals as indicated (FIG. 2A). Each mice was received 100 μl of total 100 μg peptide vaccine mixed with ISA 720 (v:v=1:1). The mice were boosted twice with three weeks intervals. Blood were collected weekly to monitor the antibody titers after the immunization. Two weeks after the last boost, all the mice were challenged with designed cancer cells as indicated in the figures, CT26 colon carcinoma cells of 1×10⁵ cells per mouse, or 4T1 breast cancer cells of 1×10⁵ cells per mouse, or D2F2 mammary carcinoma cells of 2×10⁵ cells per mouse. The tumor growths were monitored and checked daily post day 7 of tumor challenge. Tumor size was measured with calipers. For the positive control group, the mice started to treated with anti-mouse CTLA-4 monoclonal antibody (clone 9H10) twice per week, while the negative control group mice were received PBS twice per week. All the immunized mice did not received additional treatment after tumor challenge (FIG. 2A). The mice bleeds were collected weekly after the first boost until the tumor challenge at 3Y+2. The ELISA results indicated all the antibodies with high titers are over 1 to 100 thousands at the time of tumor challenge (FIG. 2B). The percentage of antibody subclass as indicated in FIG. 2C. The anti-MVF-CTLA-4 (59) with 65% of IgG1, followed by 14% of IgG2a and 7% of IgG2b. While all the other three antibodies with similar percentages of antibody subtypes, IgG1 (from 29% to 40%), IgG2a (from 23% to 29%) and IgG2b (from 29% to 39%) The antigenicity against recombinant human CTLA-4 protein indicated anti-MVF-CTLA-4 (92) with the highest absorbance value at 415 nm, followed by anti-MVF-CTLA-4 (75), anti-MVF-CTLA-4 (130), and anti-MVF-CTLA-4 (59) (FIG. 2D). The majority of tumor growths in the PBS groups were fastest than the immunized mice or mice treated with mAb (FIG. 2E).

### c) CTLA-4 Peptide Vaccines in CT26-BALB/c Tumor Model

6-8 weeks old BALB/c mice were vaccinated with MVF-CTLA-4 (59), MVF-CTLA-4 (75), MVF-CTLA-4 (92) or MVF-CTLA-4 (130) for 3 weeks interval. 0.1 mg peptide cancer vaccine mixed with ISA 720 (1:1) used per mouse. Mice were boosted with the designed doses for every 3 weeks intervals. Blood was collected weekly for monitoring antibody titers.

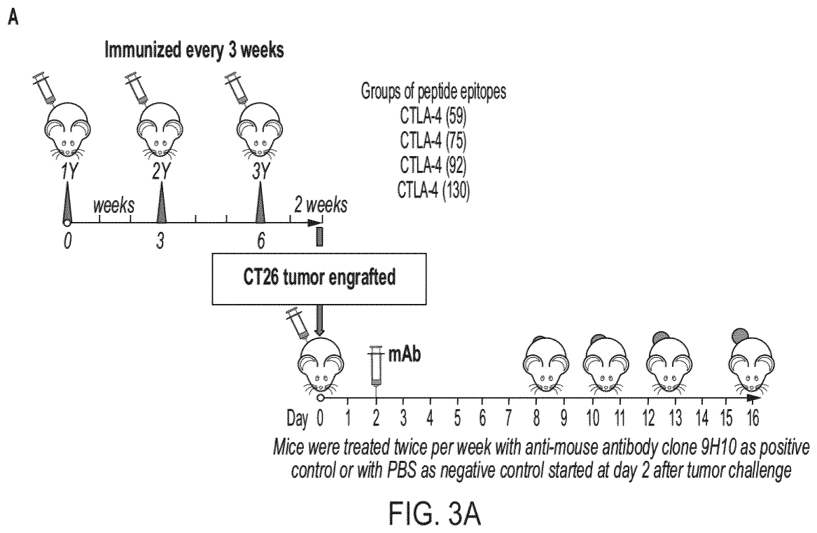

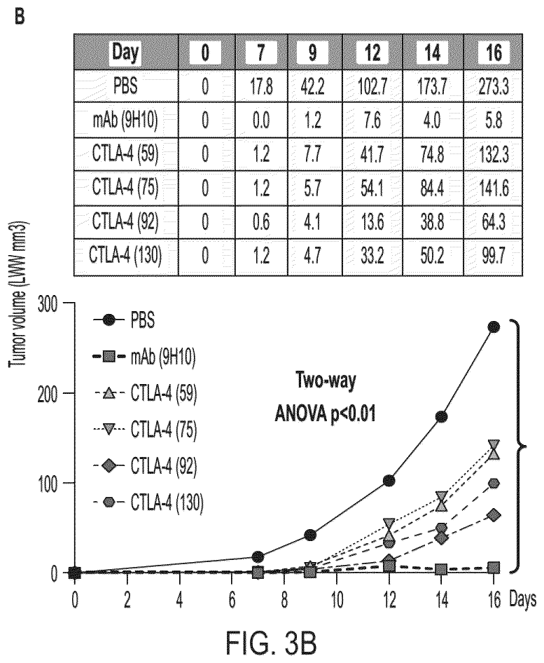

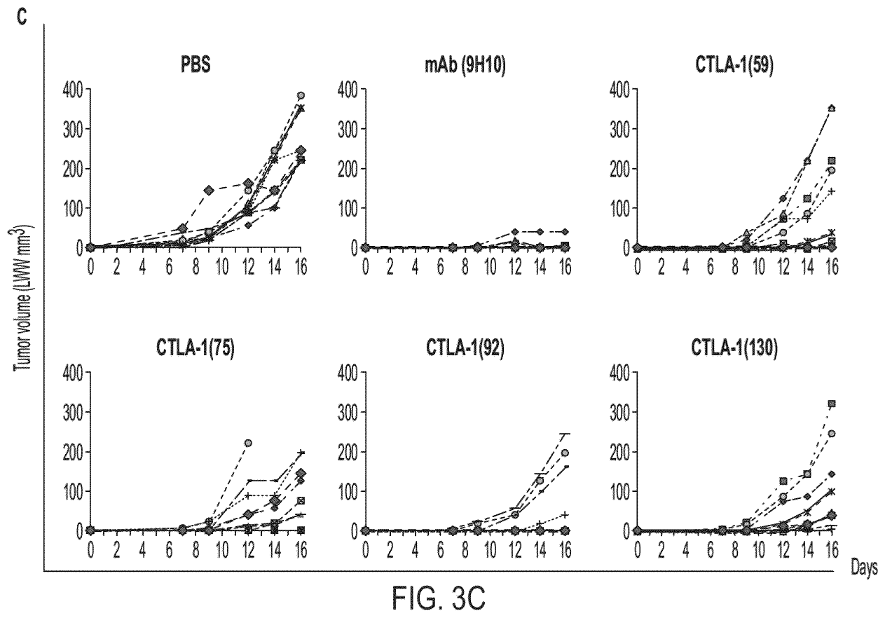

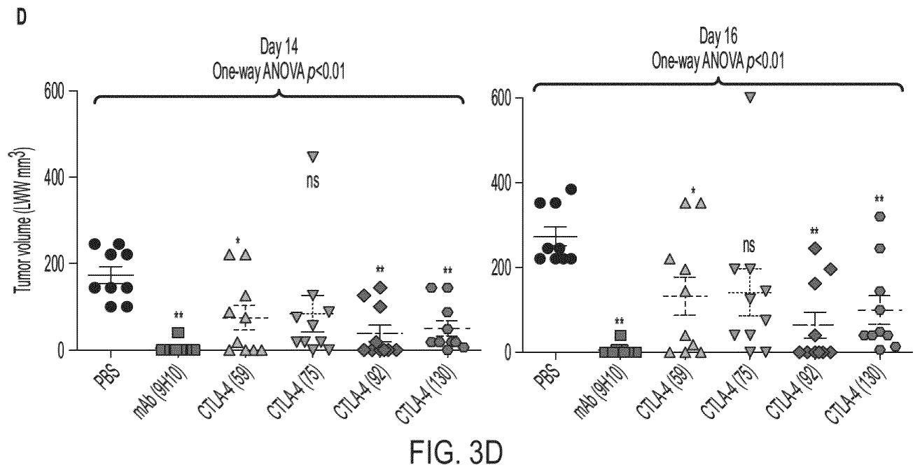

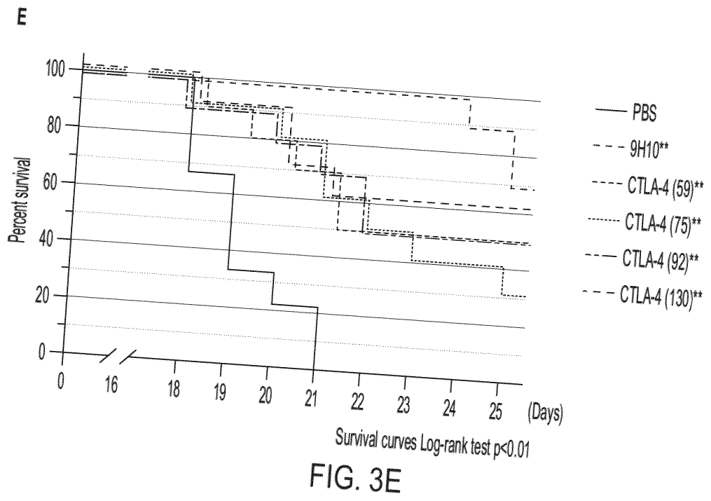

After 2 weeks of the third time immunization (3Y), mice were challenged with 10⁵ per mouse CT26 (FIG. 3A). After tumor challenge, the positive control group, we treat the mice with anti-mouse CTLA-4 antibody (clone 9H10) twice a week for up to three weeks, and the negative control group was treated with PBS. Tumor volume was calculated as: Tumor volume (LWW)=(Length×Width×Width)/2. Mean value of tumor growths in BALB/c mice were vaccinated with MVF-CTLA-4 (59), MVF-CTLA-4 (75), MVF-CTLA-4 (92) or MVF-CTLA-4 (130), PBS as negative control and anti-mouse CTLA-4 antibody (clone 9H10) as positive control. Two-way ANOVA was used to analyze the whole curves of tumor growth, which shows significant difference with p<0.01 (FIG. 3B). Individual mouse CT26 tumor growths in each group of mice were vaccinated with MVF-CTLA-4 (59), MVF-CTLA-4 (75), MVF-CTLA-4 (92) or MVF-CTLA-4 (130), PBS as negative control and anti-mouse CTLA-4 antibody (clone 9H10) as positive control (FIG. 3C).

Plots of tumor volume LWW at day 14 for each of the four treatment immunized groups together with PBS control and mAb (9H10) groups; one-way ANOVA was used to analysis multiple groups comparison, which indicated both of p<0.01. (FIG. 3D). Between groups versus with PBS group, mice in mAb (9H10), CTLA-4 (59), CTLA-4 (92) and CTLA-4 (130) group with significantly smaller tumor size versus with PBS group.

Plots of tumor volume LWW at day 16 for each of the four treatment immunized groups together with PBS control and mAb (9H10) groups; one-way ANOVA was used to analysis multiple groups comparison, which indicated both of p<0.01. Between groups versus with PBS group, mice in mAb (9H10), CTLA-4 (59), CTLA-4 (92) and CTLA-4 (130) group with significantly smaller tumor size versus with PBS group. ** indicates p<0.01, * indicates p<0.05, ns indicates no significant difference.

6-8 weeks old BALB/c mice were vaccinated with MVF-CTLA-4 (59), MVF-CTLA-4 (75), MVF-CTLA-4 (92) or MVF-CTLA-4 (130) for 3 weeks interval with two boost. Mice were challenged with 10⁵ per mouse CT26 tumor cells. (FIG. 3E) After tumor challenge, the positive control group, we treat the mice with anti-mouse CTLA-4 antibody (clone 9H10) twice a week for up to three weeks, and the negative control group was treated with PBS. The survival curves Log-rank test for multiple groups p<0.01. Between groups comparison of each individual group versus PBS group p<0.01 as indicated with **. ** indicates p<0.01;

### d) CTLA-4 Peptide Vaccines in 4T1-BALB/c Tumor Model

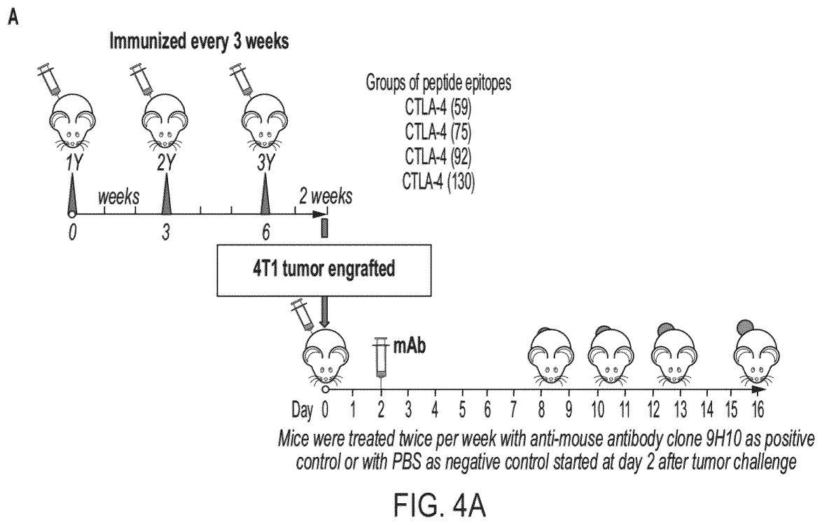

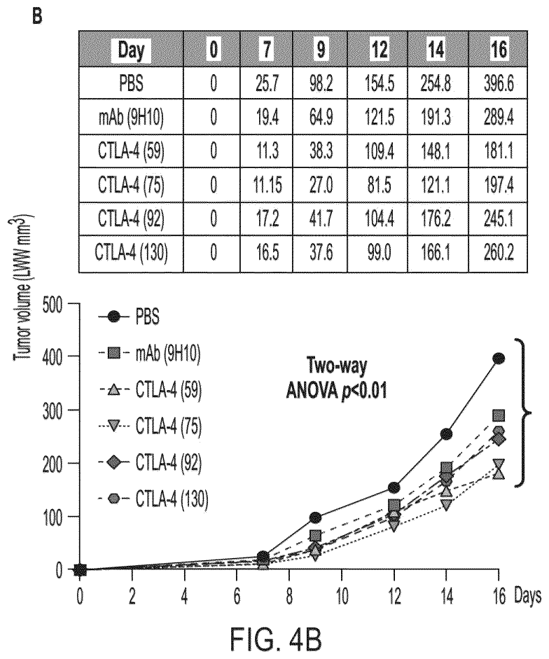

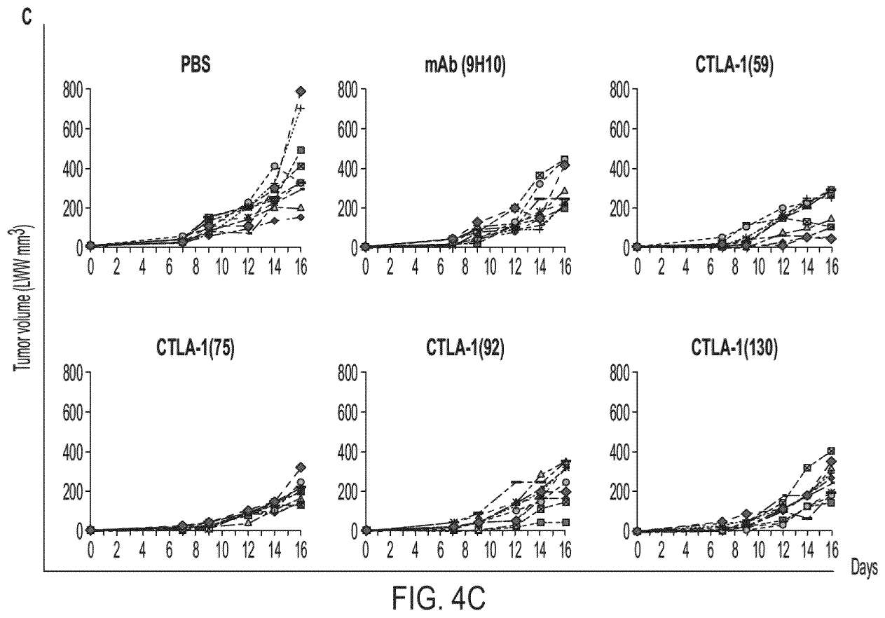

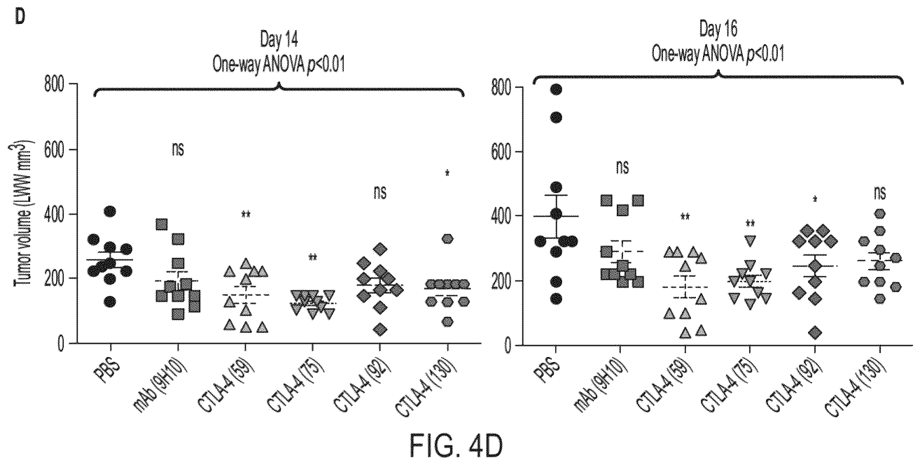

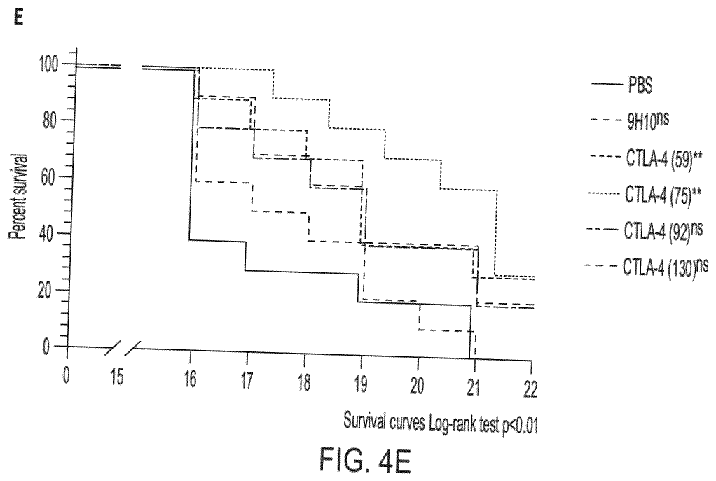

6-8 weeks old BALB/c mice were vaccinated with MVF-CTLA-4 (59), MVF-CTLA-4 (75), MVF-CTLA-4 (92) or MVF-CTLA-4 (130) for 3 weeks interval. 0.1 mg peptide cancer vaccine mixed with ISA 720 (1:1) used per mouse. Mice were boosted with the designed doses for every 3 weeks intervals. Blood was collected weekly for monitoring antibody titers. After 2 weeks of the third time immunization (3Y), mice were challenged with 10⁵ per mouse 4T1 (FIG. 4A). After tumor challenge, the positive control group, we treat the mice with anti-mouse CTLA-4 antibody (clone 9H10) twice a week for up to three weeks, and the negative control group was treated with PBS. Tumor volume was calculated as: Tumor volume (LWW)=(Length×Width×Width)/2

Mean value of tumor growths in BALB/c mice were vaccinated with MVF-CTLA-4 (59), MVF-CTLA-4 (75), MVF-CTLA-4 (92) or MVF-CTLA-4 (130), PBS as negative control and anti-mouse CTLA-4 antibody (clone 9H10) as positive control. (FIG. 4B). Two-way ANOVA was used to analyze the whole curves of tumor growth, which shows significant difference with p<0.01. Individual mouse 4T1 tumor growths in each group of mice were vaccinated with MVF-CTLA-4 (59), MVF-CTLA-4 (75), MVF-CTLA-4 (92) or MVF-CTLA-4 (130), PBS as negative control and anti-mouse CTLA-4 antibody (clone 9H10) as positive control (FIG. 4C).

Plots of tumor volume LWW at day 14 for each of the four treatment immunized groups together with PBS control and mAb (9H10) groups; one-way ANOVA was used to analysis multiple groups comparison, which indicated both of p<0.01 (FIG. 4D). Between groups versus with PBS group, mice in CTLA-4 (59), CTLA-4 (75) and CTLA-4 (130) group with significantly smaller tumor size versus with PBS group.

Plots of tumor volume LWW at day 16 for each of the four treatment immunized groups together with PBS control and mAb (9H10) groups; one-way ANOVA was used to analysis multiple groups comparison, which indicated both of p<0.01. Between groups versus with PBS group, mice in CTLA-4 (59), CTLA-4 (75) and CTLA-4 (92) group with significantly smaller tumor size versus with PBS group. ** indicates p<0.01, * indicates p<0.05, ns indicates no significant difference;

6-8 weeks old BALB/c mice were vaccinated with MVF-CTLA-4 (59), MVF-CTLA-4 (75), MVF-CTLA-4 (92) or MVF-CTLA-4 (130) for 3 weeks interval with two boost (FIG. 4E). Mice were challenged with 10⁵ per mouse 4T1 tumor cells. After tumor challenge, the positive control group, we treat the mice with anti-mouse CTLA-4 antibody (clone 9H10) twice a week for up to three weeks, and the negative control group was treated with PBS. The survival curves Log-rank test for multiple groups p<0.01. Between groups comparison of each individual group versus PBS group p<0.05 as indicated with *. * indicates p<0.05; ns indicates no significant difference;

### e) CTLA-4 Peptide Vaccines in D2F2-BALB/c Tumor Model

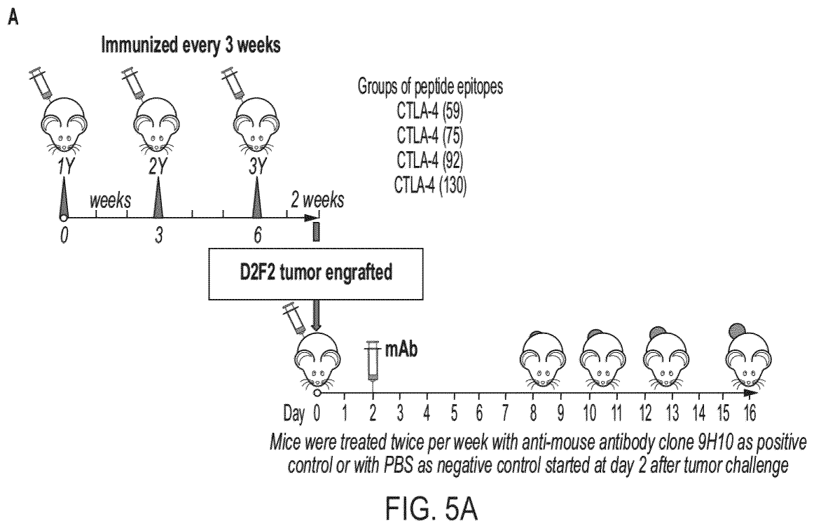

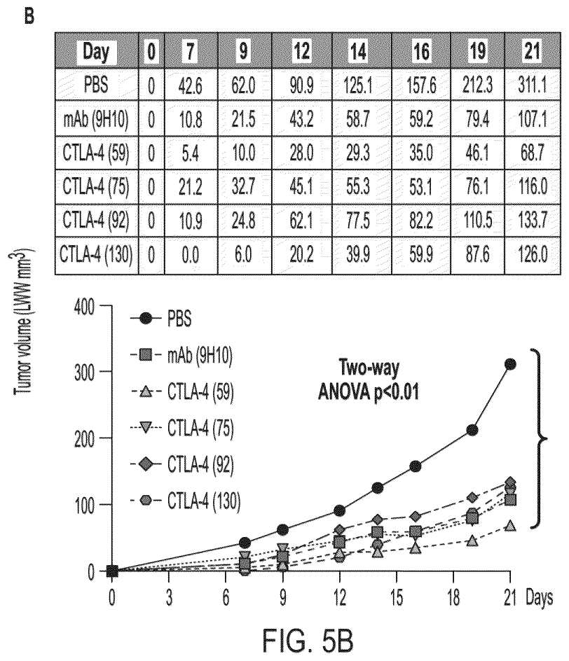

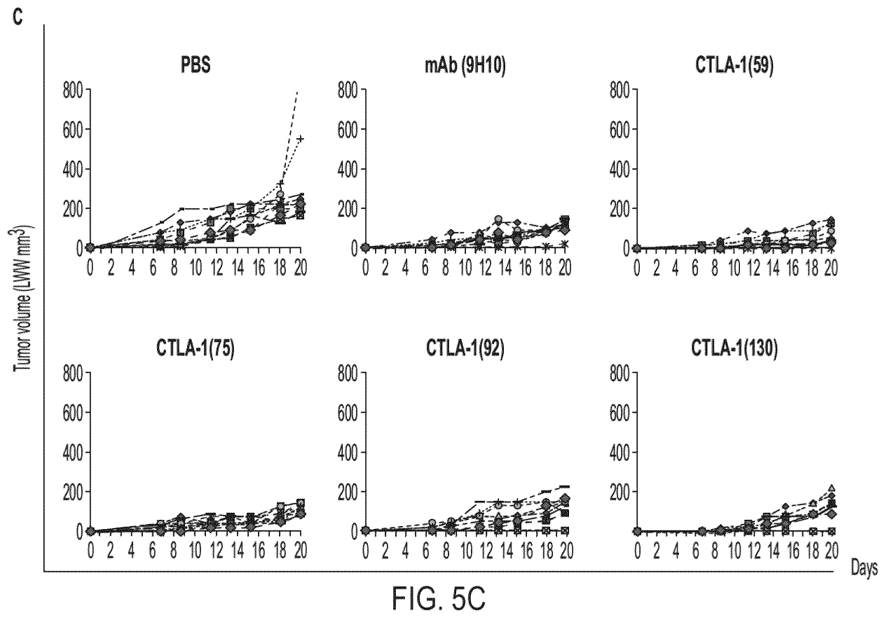

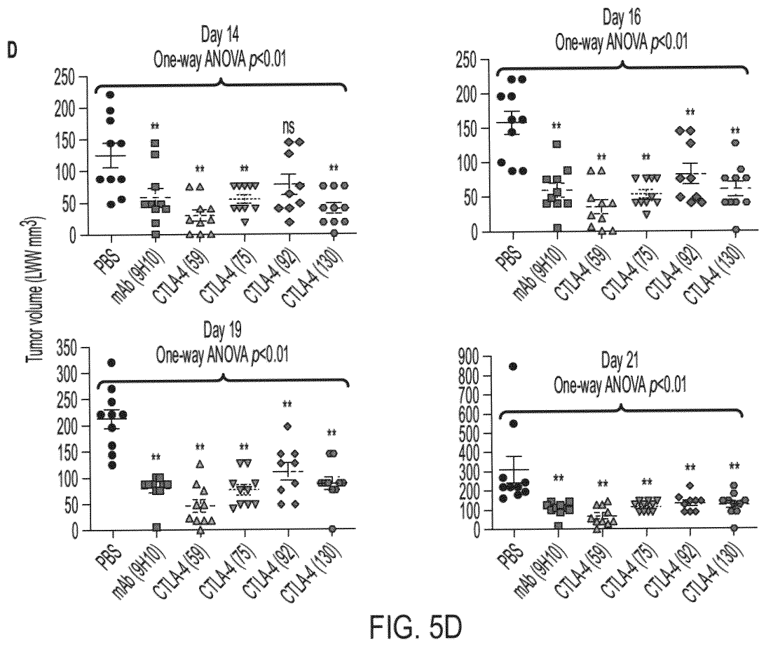

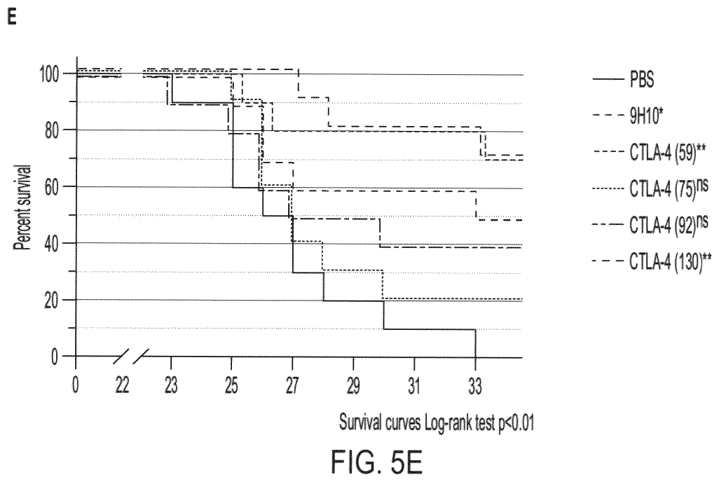

6-8 weeks old BALB/c mice were vaccinated with MVF-CTLA-4 (59), MVF-CTLA-4 (75), MVF-CTLA-4 (92) or MVF-CTLA-4 (130) for 3 weeks interval. 0.1 mg peptide cancer vaccine mixed with ISA 720 (1:1) used per mouse (FIG. 5A). Mice were boosted with the designed doses for every 3 weeks intervals. Blood was collected weekly for monitoring antibody titers. After 2 weeks of the third time immunization (3Y), mice were challenged with 2×10⁵ per mouse D2F2 mammary tumor cells.

After tumor challenge, the positive control group, we treat the mice with anti-mouse CTLA-4 antibody (clone 9H10) twice a week for up to three weeks, and the negative control group was treated with PBS. Tumor volume was calculated as: Tumor volume (LWW)=(Length×Width×Width)/2.

Mean value of tumor growths in BALB/c mice were vaccinated with MVF-CTLA-4 (59), MVF-CTLA-4 (75), MVF-CTLA-4 (92) or MVF-CTLA-4 (130), PBS as negative control and anti-mouse CTLA-4 antibody (clone 9H10) as positive control (FIG. 5B). Two-way ANOVA was used to analyze the whole curves of tumor growth, which shows significant difference with p<0.01.

Individual mouse D2F2 tumor growths in each group of mice were vaccinated with MVF-CTLA-4 (59), MVF-CTLA-4 (75), MVF-CTLA-4 (92) or MVF-CTLA-4 (130), PBS as negative control and anti-mouse CTLA-4 antibody (clone 9H10) as positive control (FIG. 5C).

Plots of tumor volume LWW at day 14 for each of the four treatment immunized groups together with PBS control and mAb (9H10) groups; one-way ANOVA was used to analysis multiple groups comparison, which indicated both of p<0.01 (FIG. 5D). Between groups versus with PBS group, mice in mAb (9H10), CTLA-4 (59), CTLA-4 (75) and CTLA-4 (130) group with significantly smaller tumor size versus with PBS group.

Plots of tumor volume LWW at day 16 for each of the four treatment immunized groups together with PBS control and mAb (9H10) groups; one-way ANOVA was used to analysis multiple groups comparison, which indicated both of p<0.01. Between groups versus with PBS group, mice in mAb (9H10), CTLA-4 (59), CTLA-4 (75), CTLA-4 (92) and CTLA-4 (130) group with significantly smaller tumor size versus with PBS group.

Plots of tumor volume LWW at day 19 for each of the four treatment immunized groups together with PBS control and mAb (9H10) groups; one-way ANOVA was used to analysis multiple groups comparison, which indicated both of p<0.01. Between groups versus with PBS group, mice in mAb (9H10), CTLA-4 (59), CTLA-4 (75), CTLA-4 (92) and CTLA-4 (130) group with significantly smaller tumor size versus with PBS group.

Plots of tumor volume LWW at day 21 for each of the four treatment immunized groups together with PBS control and mAb (9H10) groups; one-way ANOVA was used to analysis multiple groups comparison, which indicated both of p<0.01. Between groups versus with PBS group, mice in mAb (9H10), CTLA-4 (59), CTLA-4 (75), CTLA-4 (92) and CTLA-4 (130) group with significantly smaller tumor size versus with PBS group. ** indicates p<0.01, * indicates p<0.05, ns indicates no significant difference. 133. 6-8 weeks old BALB/c mice were vaccinated with MVF-CTLA-4 (59), MVF-CTLA-4 (75), MVF-CTLA-4 (92) or MVF-CTLA-4 (130) for 3 weeks interval with two boost (FIG. 5E). Mice were challenged with 2×10⁵ per mouse D2F2 tumor cells. After tumor challenge, the positive control group, we treat the mice with anti-mouse CTLA-4 antibody (clone 9H10) twice a week for up to three weeks, and the negative control group was treated with PBS. The survival curves Log-rank test for multiple groups p<0.01. Between groups comparison of each individual group versus PBS group p<0.05 as indicated. ** indicates p<0.01, * indicates p<0.05; ns indicates no significant difference.

### f) CTLA-4 Peptide Mimics in CT26-BALB/c Tumor Model (V32)

### (1) Schematic of CTLA-4 Peptide Epitopes Therapeutic Experiment after Mice were Challenged with CT26 Tumor Cells

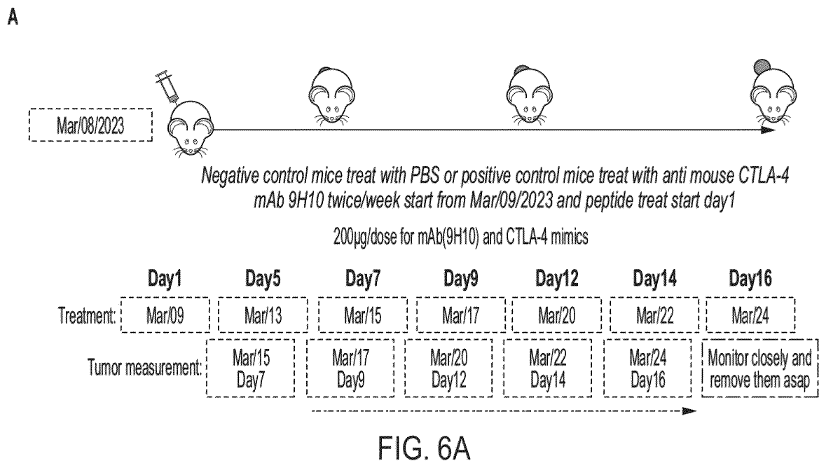

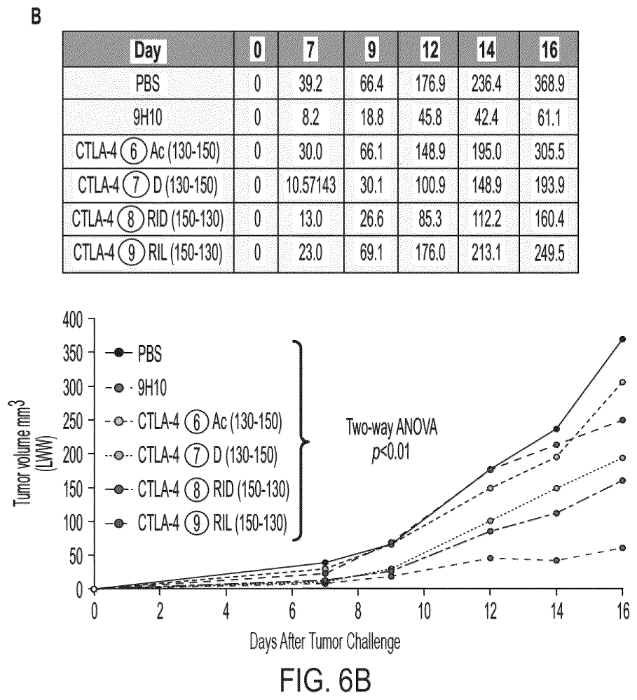

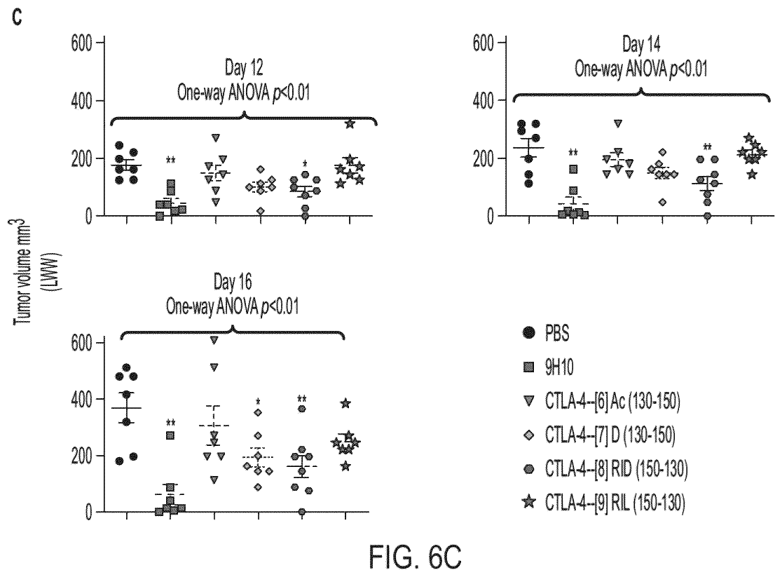

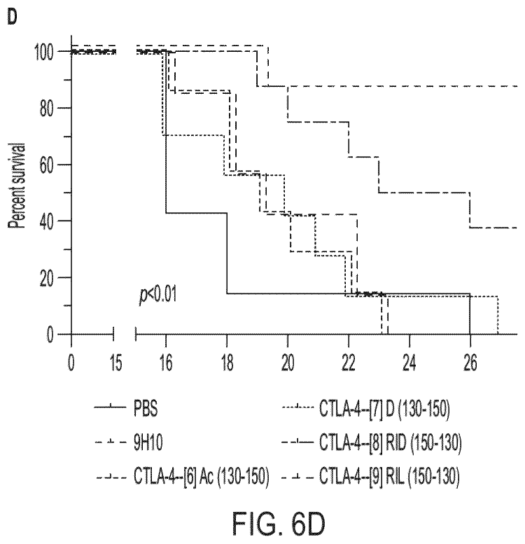

6-8 weeks old BALB/c mice were challenged with 1×10⁵ per mouse CT26 tumor cells (FIG. 6A). After tumor challenge, the positive control group, we treat the mice with anti-mouse CTLA-4 antibody (clone 9H10) twice a week, start from day 1 post tumor challenge and the negative control group was treated with PBS. For the CTLA-4 peptide mimic treatment groups, mice were treated with 200 ug peptide mimics as indicated start from day 1 post tumor challenging. All the mice had been treated at day 1, day 2, day 5, day 7, day 9, day 12, day 14, and day 16 post tumor challenge. The immunogenicity (antibody titer) was under detectable. Tumor volume was calculated as: Tumor volume (LWW)=(Length×Width×Width)/2

### (2) Mean Value of Tumor Growths in BALB/c Mice

BALB/c mice treated with CTLA-4 peptide mimics or anti-mouse CTLA-4 antibody as positive control, PBS as negative control (FIG. 6B). Two-way ANOVA was used to analyze the whole curves of tumor growth, which shows significant difference with p<0.01;

### (3): Tumor Volume Comparisons

Due to 6/10 PBS mice were removed on Day 14, two more mice were removed on Day 15, all the mice in PBS group were gone on Day 16, so no further tumor volume comparisons after Day 14 (FIG. 6C).

### (a) At Day9

One-way ANOVA p<0.01. Between groups comparisons all the treatment groups vs PBS with significant difference.

### (b) At Day 12

One-way ANOVA p<0.01. Between groups comparisons only 9H10 vs PBS with p<0.01, all the other treatment groups without statistics difference compared with PBS.

### (c) At Day 14

One-way ANOVA p<0.01. Between groups comparisons: 9H10 vs PBS with p<0.01, CTLA-4 \textcircled{9} RIL (150-130) vs PBS p<0.05. All the left treatment groups without statistics difference compared with PBS. Survival curves comparison Log-rank (Mantel-Cox) test (FIG. 6D). Between groups comparisons all the treatment groups vs PBS with significant difference. * indicates p<0.05; ** indicates p<0.01.

### g) Generalized Statistics Analysis of Antitumor Efficacy in Syngeneic BALB/c Mice Challenged with CT26 Colon Carcinoma, 4T1 and D2F2 Mammary Cancer Cell Lines

In the three syngeneic BALB/c mice tumor models, CT26 colon carcinoma, 4T1 and D2F2 mammary tumor models, two-way ANOVA analysis indicated the tumor growth with significant different rate in the different groups. In the CT26 colon carcinoma tumor model, the anti-mouse monoclonal antibody (clone 9H10) group, MVF-CTLA-4 (59), MVF-CTLA-4 (92), and MVF-CTLA-4 (130) immunized groups with significant smaller tumor size both at day 14 and day 16 versus with PBS group. In the 4T1 breast cancer model, MVF-CTLA-4 (59), MVF-CTLA-4 (75), and MVF-CTLA-4 (130) immunized group with significant smaller tumor size at day 14, while at day 16 the significant smaller tumor size groups were MVF-CTLA-4 (59), MVF-CTLA-4 (75), and MVF-CTLA-4 (92) immunized groups, all compared with PBS group. In the D2F2 mammary tumor model, all the treatment groups with significant less tumor burden at day 14, day 16, day 19 and day 21, except MVF-CTLA-4 (92) immunized group at day 14 without difference with PBS group

Longer survival rate is the most important thing for the cancer patients. In the CT26 colon carcinoma tumor model, the anti-mouse monoclonal antibody (clone 9H10) group, together with all the immunized groups with significant longer survival rate versus with PBS group. In the 4T1 breast cancer model, only MVF-CTLA-4 (59), and MVF-CTLA-4 (75) immunized group with significant longer survival compared with PBS group. In the D2F2 mammary tumor model, anti-mouse monoclonal antibody (clone 9H10) group together with MVF-CTLA-4 (59), and MVF-CTLA-4 (130) immunized group with significant different longer survival than PBS group.

### h) Methods

### (1) Peptide Synthesis, HPLC and Characterization (SEE CTLA-4 Peptide Binder)

All CTLA-4 peptides were commercially synthesized by Mimotopes (Australia) and the CTLA-4 peptides were synthesized by solid phase peptide synthesis. Chimeric CTLA-4 B-cell peptides vaccines were made by adding a measles virus fusion peptide (MVF, amino acids 288-302, KLLSLIKGVIVHRLEGVE) with a four amino acid residue (GPSL) to the CTLA-4 peptides. Briefly, four novel peptide sequences targeting B-cell epitopes of human CTLA-4 were identified and synthesized using a 9600 Milligen/Biosearch solid-phase peptide synthesizer (Millipore, Bedford, MA, USA) with Fmoc/t-Butyl chemistry and PyBOP/6Cl-HOBT coupling reagents on CLEAR amide resin (Peptides International, Louisville, KY, USA). Some peptide samples were acetylated using 1-Acetylimidazole (Sigma-Aldrich St. Louis, MO, USA) before cleavage to provide samples of stable peptide B-cell epitopes. Each of the four peptides were synthesized as chimeric constructs with “promiscuous” T helper epitope (MVF). Peptides were cleaved from the peptide resin using cleavage reagent R (TFA)/thioanisole/EDT/anisole (90/5/3/2). Crude peptides were purified by semi preparative (C-4 Vydac columns) reverse-phase-high performance liquid chromatography (RP-HPLC; Waters, Bedford, MA, USA). HPLC fractions collected at various times with the same retention time were pooled together and lyophilized. All peptides showed purity in excess of 95%. Samples were then characterized by MALDI (Matrix-Assisted Laser Desorption Ionization) mass spectroscopy at the CCIC (Campus Chemical Instrumentation Center, The Ohio State University, Columbus, OH, USA) and analyzed on an analytical RP-HPLC system (Waters, Bedford, MA, USA). All peptides had the correct molecular mass.

### (2) Animals: Rabbits and BALB/c Mice

All experiments were performed in accordance with the U.S. Public Health Service Policy on Humane Care and Use of Laboratory Animals and approved by the Ohio State University Institutional Animals Care and Use Committee and detailed in the approved protocol. New Zealand white female rabbits and BALB/c female mice were purchased from Charles River Laboratories (Wilmington, MA, USA). All animal care and use was in accordance with ULAR (University Laboratory Animal Resources) institutional guidelines.

### (3) Animal Immunization

For each peptide, vaccine antibodies were raised using female New Zealand white rabbits (>2 Kg/8-10 weeks of age) purchased from Charles River Laboratories (Wilmington, MA, USA). Rabbits were immunized with 1 mg chimeric MVF linked CTLA-4 peptides and boosted twice at three weeks and at six weeks. BALB/c mice were immunized with 100 μg MVF linked peptides. The four chimeric peptide based candidate vaccines were used to immunize all animals. BALB/c female mice (5-6 weeks old) were immunized with chimeric peptide immunogens 3 times at 3 week intervals referred to as primary immunization (1Y), first boost (2Y) and second boost (3Y). The mice sera were collected every week after secondary and tertiary immunization (2Y, 2Y+1, 2Y+2, 3Y, 3Y+1 and 3Y+2), and stored at −20° C. for future use.

### (4) Cell Lines

CT26 wild type (CT26 WT) and 4T1 tumor cell lines were purchased from ATCC (Manassas, VA, USA). Mouse mammary carcinoma cell line D2F2 wild type was kindly provided by Professor Wei-Zen Wei (Karmanos Cancer Institute, Wayne State University School of Medicine, Detroit, Michigan, USA). D2F2 is syngeneic to BALB/c mice murine mammary tumor cells. CT26 WT and 4T1 cell lines were maintained in DMEM/RPMI-1640 basic medium. D2F2 cell line was maintained in DMED with 10% NCTC-109 medium (Invitrogen, Waltham, MA, USA) and 1×MEM Non-Essential Amino Acids Solution (ThermoFisher, Rockford, IL, USA). All cell culture media were supplied with 10% fetal bovine serum (FBS), 100 units/ml penicillin and 100 μg/ml streptomycin.

### (5) Enzyme-Linked Immunosorbent Assay (ELISA)

Immunogenicity was evaluated by ELISA as per our laboratory standard protocols. Briefly, 96-well plates were coated with 100 μl of peptide as antigen at 2 μg/ml in PBS overnight at 4° C. Nonspecific binding sites were blocked for 1 h with 200 μl PBS (Research Products International, Mt Prospect, IL, USA, CAS No. 7647-145) 1% BSA (Bovine serum albumin, Thermo Fisher Scientific, Waltham, WA, USA, BP9703-100), and plates were washed with washing buffer (PBS diluted 0.05% Tween 1% horse serum). Vaccine antibodies in blocking buffer (PBS 1% BSA) were added to antigen-coated plate in duplicate wells, serially diluted 1:2 in blocking buffer, and incubated for 2 h at room temperature. After washing the plate, the secondary antibody 100 μl of 1:500 goat anti-mouse IgG conjugated to horseradish peroxidase (Invitrogen, Waltham, MA, USA, REF: 31430) were added to each well and incubated for 1 h. After washing, the antibody was detected using substrate solution (50 μl of 0.15% H₂O₂ in 24 mM citric acid and 5 mM sodium phosphate buffer (pH 5.2) with 0.5 mg/ml 2,2′-aminobis (3-ethylbenzthiazole-6-sulfonic acid, ABTS, Sigma, St. Louis, MO, USA) as the chromophore. Color development proceeded for 10 min, and the reaction was stopped with 25 μl of 1% SDS (sodium dodecyl sulfate, Thermo Scientific, Waltham, WA, USA, Prod #28312). Absorbance was read at 415 nm using an ELISA Microplate reader (Molecular Devices, SPECTRAmax PLUS384, San Jose, CA, USA).

### (6) Recombinant Protein Activity Assay

For the detection of antibody reactivity with human CTLA-4 recombinant protein (CTLA-4, CT4-H5229, HIS tag, ACROBiosystems, Newark, DE, USA) lug recombinant protein in 100 μl of PBS or the concentration as indicated in the figures was used to coat wells overnight at 4° C. After the overnight incubation, nonspecific binding sites were blocked for 1 h with 200 μl PBS 1% BSA, and plates were washed with washing buffer (PBS diluted 0.05% Tween 1% horse serum). Vaccine antibodies in blocking buffer were added to antigen-coated plate in duplicate wells, serially diluted 1:2 in blocking buffer, and incubated for 2 h at room temperature. After washing the plate, 100 μl of 1:500 goat anti-mouse IgG conjugated to horseradish peroxidase (Invitrogen, Waltham, MA, USA, REF: 31430) were added to each well and incubated for 1 h. The plate received a final wash and 50 μl prepared substrate solution was added to each well (BIO-RAD, Hercules, CA, USA, Cat. #1721064). The reaction was stopped with 25 μl 5% SDS stopping buffer. Absorbance at 415 nm was determined using a plate reader.

### (7) Antibody Isotyping Assay

The assay was carried out by following the manufacturer's instructions (BIO-RAD, Mouse Typer isotyping kit, Cat. #172-2055) and lab protocol. Briefly, mouse antibody isotypes (i.e. IgA, IgM, IgG1, IgG2a, IgG2b, and IgG3) were determined using the Mouse Typer isotyping Kit (BIO-RAD, Hercules, CA, USA, Cat. #172-2055). Briefly, wells of a 96-well assay plate (COSTAR, Washington, D.C., USA, REF #2797) were coated with 100 μl of 2 μg/ml peptide antigen in ddH₂O, and incubated at 4° C. overnight. The plate was washed with washing buffer (0.05% tween-20 and 1% horse sera in PBS). The plate was blocked with 1% BSA in PBS at room temperature for 1 h. 100 μl of diluted sera was added to each well. Dilutions of each sera samples were determined by the ELISA titers absorbance of 0.4 or higher after subtracting the background. After washing the wells, 100 μl ready to use rabbit anti-mouse subclasses antibodies were added to each well respectively and incubated at room temperature for 2 h. The wells were washed again, 100 μl (1/3000 dilution of goat anti-rabbit conjugated to HRP antibody (BIO-RAD, Hercules, CA, USA, Cat. #172-1019)) was added to each well and incubated for 1 h at room temperature in dark. The plate received a final wash and 50 μl prepared substrate solution was added to each well (BIO-RAD, Hercules, CA, USA, Cat. #1721064). The reaction was stopped with 25 μl 5% SDS stopping buffer. Absorbance at 415 nm was determined using a plate reader.

### (8) Statistical Analysis

Mice challenged with tumor cells were monitored at least twice per week and tumor sizes were measured by calipers. Formula: Volume (LWW)=(Length×Width×Width)/2 was used to calculate tumor volumes. All values are showed as means±standard deviation. Data statistical analysis was performed by GraphPad Prism 8.1.2 (GraphPad Software, Inc. San Diego, CA, USA) and the indicated statistical analysis. One-way analysis of variance (one-way ANOVA) and followed by the Tukey's multiple comparisons test were used to compare data in multiple groups or data between groups in multiple groups. And the two-way ANOVA was used to analysis the whole curves comparison. The Log-rank (Mantel-Cox) test was use to compare the survival curves. P value or adjusted p value less than 0.05 was accepted as statistically significant different.

### E. REFERENCES

Allen S D, Garrett J T, Rawale S V, Jones A L, Phillips G, Forni G, Morris J C, Oshima R G, Kaumaya P T: Peptide vaccines of the HER-2/neu dimerization loop are effective in inhibiting mammary tumor growth in vivo. Journal of immunology (Baltimore, Md: 1950) 2007, 179 (1): 472-482.
Allen S D, Rawale S V, Whitacre C C, Kaumaya P T: Therapeutic peptidomimetic strategies for autoimmune diseases: costimulation blockade. J Pept Res 2005, 65 (6): 591-604.
Arteaga C L, Engelman J A: ERBB receptors: from oncogene discovery to basic science to mechanism-based cancer therapeutics. Cancer cell 2014, 25 (3): 282-303.
Baselga J, Arteaga C L: Critical update and emerging trends in epidermal growth factor receptor targeting in cancer. J Clin Oncol 2005, 23 (11): 2445-2459.
Baselga J, Swain S M: Novel anticancer targets: revisiting ERBB2 and discovering ERBB3. Nature reviews Cancer 2009, 9 (7): 463-475.
Baselga J: Targeting tyrosine kinases in cancer: the second wave. Science 2006, 312 (5777): 1175-1178.
Buchbinder E, Hodi F S: Cytotoxic T lymphocyte antigen-4 and immune checkpoint blockade. J Clin Invest 2015, 125 (9): 3377-3383.
Chames P, Van Regenmortel M, Weiss E, Baty D: Therapeutic antibodies: successes, limitations and hopes for the future. Br J Pharmacol 2009, 157 (2): 220-233.
Chou P Y, Fasman G D: Prediction of the secondary structure of proteins from their amino acid sequence. Advances in enzymology and related areas of molecular biology 1978, 47:45-148.
Cobleigh M A, Langmuir V K, Sledge G W, Miller K D, Haney L, Novotny W F, Reimann J D, Vassel A: A phase I/II dose-escalation trial of bevacizumab in previously treated metastatic breast cancer. Seminars in oncology 2003, 30 (5 Suppl 16): 117-124.
Dakappagari N K, Douglas D B, Triozzi P L, Stevens V C, Kaumaya P T: Prevention of mammary tumors with a chimeric HER-2 B-cell epitope peptide vaccine. Cancer Res 2000, 60 (14): 3782-3789.
Dakappagari N K, Lute K D, Rawale S, Steele J T, Allen S D, Phillips G, Reilly R T, Kaumaya P T: Conformational HER-2/neu B-cell epitope peptide vaccine designed to incorporate two native disulfide bonds enhances tumor cell binding and antitumor activities. J Biol Chem 2005, 280 (1): 54-63.
Dakappagari N K, Pyles J, Parihar R, Carson W E, Young D C, Kaumaya P T: A chimeric multi-human epidermal growth factor receptor-2 B cell epitope peptide vaccine mediates superior antitumor responses. J Immunol 2003, 170 (8): 4242-4253.
Dakappagari N K, Sundaram R, Rawale S, Liner A, Galloway D R, Kaumaya P T: Intracellular delivery of a novel multiepitope peptide vaccine by an amphipathic peptide carrier enhances cytotoxic T-cell responses in HLA-A*201 mice. J Pept Res 2005, 65 (2): 189-199.
Darvin P, Toor S M, Sasidharan Nair V, Elkord E: Immune checkpoint inhibitors: recent progress and potential biomarkers. Exp Mol Med 2018, 50 (12): 1-11.
Eskens F A, Verweij J: The clinical toxicity profile of vascular endothelial growth factor (VEGF) and vascular endothelial growth factor receptor (VEGFR) targeting angiogenesis inhibitors; a review. European journal of cancer 2006, 42 (18): 3127-3139.
Folkman J: Tumor angiogenesis: therapeutic implications. The New England journal of medicine 1971, 285 (21): 1182-1186.
Foy K C, Liu Z, Phillips G, Miller M, Kaumaya P T: Combination treatment with HER-2 and VEGF peptide mimics induces potent anti-tumor and anti-angiogenic responses in vitro and in vivo. J Biol Chem 2011, 286 (15): 13626-13637.
Foy K C, Miller M J, Moldovan N, Carson W E, 3rd, Kaumaya P T: Combined vaccination with HER-2 peptide followed by therapy with VEGF peptide mimics exerts effective anti-tumor and anti-angiogenic effects in vitro and in vivo. Oncoimmunology 2012, 1 (7): 1048-1060.
Foy K C, Miller M J, Moldovan N, Carson W E, Kaumaya P T P: Combined vaccination with HER-2 peptide followed by therapy with VEGF peptide mimics exerts effective anti-tumor and anti-angiogenic effects in vitro and in vivo. OncoImmunology 2012, 1 (7): 0--1.
Foy K C, Vicari D, Kaumaya P T P: Therapeutic Peptides Targeting HER-2/neu and VEGF Signaling Pathways in Breast Cancer. In: Handbook of Biologically Active Peptides. 2013:612-616.
Grothey A: Recognizing and managing toxicities of molecular targeted therapies for colorectal cancer. Oncology (Williston Park) 2006, 20 (14 Suppl 10): 21-28.
Harding F A, Stickler M M, Razo J, DuBridge R B: The immunogenicity of humanized and fully human antibodies: residual immunogenicity resides in the CDR regions. mAbs 2010, 2 (3): 256-265.
Hoeben A, Landuyt B, Highley M S, Wildiers H, Van Oosterom A T, De Bruijn E A: Vascular endothelial growth factor and angiogenesis. Pharmacol Rev 2004, 56 (4): 549-580.
Hopp T P, Woods K R: Prediction of protein antigenic determinants from amino acid sequences. Proceedings of the National Academy of Sciences of the United States of America 1981, 78 (6): 3824-3828.
Houck K A, Ferrara N, Winer J, Cachianes G, Li B, Leung D W: The vascular endothelial growth factor family: identification of a fourth molecular species and characterization of alternative splicing of RNA. Molecular endocrinology 1991, 5 (12): 1806-1814.
Hynes N E, Lane H A: ERBB receptors and cancer: the complexity of targeted inhibitors. Nature reviews Cancer 2005, 5 (5): 341-354.
Jain R K, Duda D G, Clark J W, Loeffler J S: Lessons from phase III clinical trials on anti-VEGF therapy for cancer. Nat Clin Pract Oncol 2006, 3 (1): 24-40.
Karplus P A, Schulz G E: Refined structure of glutathione reductase at 1.54 A resolution. Journal of molecular biology 1987, 195 (3): 701-729.
Kaumaya P T, Foy K C, Garrett J, Rawale S V, Vicari D, Thurmond J M, Lamb T, Mani A, Kane Y, Balint C R et al: Phase I active immunotherapy with combination of two chimeric, human epidermal growth factor receptor 2, B-cell epitopes fused to a promiscuous T-cell epitope in patients with metastatic and/or recurrent solid tumors. J Clin Oncol 2009, 27 (31): 5270-5277.
Kaumaya P T: A paradigm shift: Cancer therapy with peptide-based B-cell epitopes and peptide immunotherapeutics targeting multiple solid tumor types: Emerging concepts and validation of combination immunotherapy. Human vaccines & immunotherapeutics 2015, 11 (6): 1368-1386.
Kaumaya P T: Could precision-engineered peptide epitopes/vaccines be the key to a cancer cure? Future Oncol 2011, 7 (7): 807-810.
Kaumaya P T P K-CS, DiGeorge A M, Stevens V.: In: G. M., Basava AnantharamaiahC. editors. PEPTIDES. Boston, US: Springer-Verlag; 1994: p. 133-164.
Kaumaya P T P, Guo L, Overholser J, Penichet M L, Bekaii-Saab T: Immunogenicity and antitumor efficacy of a novel human PD-1 B-cell vaccine (PD1-Vaxx) and combination immunotherapy with dual trastuzumab/pertuzumab-like HER-2 B-cell epitope vaccines (B-Vaxx) in a syngeneic mouse model. Oncoimmunology 2020, 9 (1): 1818437.
Kaumaya P T P, Kobs-Conrad S, DiGeorge A M, Stevens V: Denovo Engineering of Protein Immunogenic & Antigenic Determinants. In: PEPTIDES. Edited by Anantharamaiah G M B, C., vol. 9: Springer-Verlag.; 1994:133-164.
Kaumaya P T P: HER-2/neu cancer vaccines: Present status and future prospects. International Journal of Peptide Research and Therapeutics 2006, 12 (1): 65-77.
Krummel M F, Allison J P: CD28 and CTLA-4 have opposing effects on the response of T cells to stimulation. J Exp Med 1995, 182 (2): 459-465.
Kyte J, Doolittle R F: A simple method for displaying the hydropathic character of a protein. Journal of molecular biology 1982, 157 (1): 105-132.
Ledford H: Melanoma drug wins US approval. Nature 2011, 471 (7340): 561.
Li B, Ogasawara A K, Yang R, Wei W, He G W, Zioncheck T F, Bunting S, de Vos A M, Jin H: KDR (VEGF receptor 2) is the major mediator for the hypotensive effect of VEGF. Hypertension 2002, 39 (6): 1095-1100.
Lynch M P, Kaumaya P T P: Advances in HTLV-1 peptide vaccines and therapeutics. Current Protein and Peptide Science 2006, 7 (2): 137-145.
Marin-Acevedo J A, Kimbrough E O, Lou Y: Next generation of immune checkpoint inhibitors and beyond. J Hematol Oncol 2021, 14 (1): 45.
Miller M J, Foy K C, Kaumaya P T: Cancer immunotherapy: present status, future perspective, and a new paradigm of peptide immunotherapeutics. Discovery medicine 2013, 15 (82): 166-176.
Miller M J, Foy K C, Kaumaya P T P: Cancer immunotherapy: Present status, future perspective, and a new paradigm of peptide immunotherapeutics. Discovery medicine 2013, 15 (82): 166-176.
Nakajima M, Tamada K: Cancer Immunotherapy Targeting Co-signal Molecules. Adv Exp Med Biol 2019, 1189:313-326.
Nelson A L, Dhimolea E, Reichert J M: Development trends for human monoclonal antibody therapeutics. Nature reviews Drug discovery 2010, 9 (10): 767-774.
Novotny J, Handschumacher M, Haber E, Bruccoleri R E, Carlson W B, Fanning D W, Smith J A, Rose G D: Antigenic determinants in proteins coincide with surface regions accessible to large probes (antibody domains). Proceedings of the National Academy of Sciences of the United States of America 1986, 83 (2): 226-230.
Oshima R G, Lesperance J, Munoz V, Hebbard L, Ranscht B, Sharan N, Muller W J, Hauser C A, Cardiff R D: Angiogenic acceleration of Neu induced mammary tumor progression and metastasis. Cancer Res 2004, 64 (1): 169-179.
Podlesnykh S V, Abramova K E, Gordeeva A, Khlebnikov A I, Chapoval A I: Peptide Blocking CTLA-4 and B7-1 Interaction. Molecules 2021, 26 (2).
Ribas A, Wolchok J D: Cancer immunotherapy using checkpoint blockade. Science 2018, 359 (6382): 1350-1355.
Robert C: A decade of immune-checkpoint inhibitors in cancer therapy. Nat Commun 2020, 11 (1): 3801.
Rose G D, Geselowitz A R, Lesser G J, Lee R H, Zehfus M H: Hydrophobicity of amino acid residues in globular proteins. Science 1985, 229 (4716): 834-838.
Roskoski R, Jr.: The ErbB/HER family of protein-tyrosine kinases and cancer. Pharmacological research: the official journal of the Italian Pharmacological Society 2014, 79:34-74.
Sharma P, Allison J P: The future of immune checkpoint therapy. Science 2015, 348 (6230): 56-61.
Srinivasan M, Gienapp I E, Stuckman S S, Rogers C J, Jewell S D, Kaumaya P T, Whitacre C C: Suppression of experimental autoimmune encephalomyelitis using peptide mimics of CD28. J Immunol 2002, 169 (4): 2180-2188.
Srinivasan M, Wardrop R M, Gienapp I E, Stuckman S S, Whitacre C C, Kaumaya P T: A retro-inverso peptide mimic of CD28 encompassing the MYPPPY motif adopts a polyproline type II helix and inhibits encephalitogenic T cells in vitro. J Immunol 2001, 167 (1): 578-585.
Steele J T, Allen S D, Kaumaya P T P: Cancer Immunotherapy with Rationally Designed Synthetic Peptides. In: Handbook of Biologically Active Peptides. 2006:491-498.
Sundaram R, Dakappagari N K, Kaumaya P T P: Synthetic peptides as cancer vaccines. Biopolymers—Peptide Science Section 2002, 66 (3): 200-216.
Thornton J M, Edwards M S, Taylor W R, Barlow D J: Location of ‘continuous’ antigenic determinants in the protruding regions of proteins. The EMBO journal 1986, 5 (2): 409-413.
Topalian S L, Drake C G, Pardoll D M: Immune checkpoint blockade: a common denominator approach to cancer therapy. Cancer cell 2015, 27 (4): 450-461.
Vicari D, Foy K C, Liotta E M, Kaumaya P T: Engineered Conformation-dependent VEGF Peptide Mimics Are Effective in Inhibiting VEGF Signaling Pathways. J Biol Chem, 286 (15): 13612-13625.
Wang B, Kaumaya P T, Cohn D E: Immunization with synthetic VEGF peptides in ovarian cancer. Gynecol Oncol 2010, 119 (3): 564-570.
Wei S C, Duffy C R, Allison J P: Fundamental Mechanisms of Immune Checkpoint Blockade Therapy. Cancer Discov 2018, 8 (9): 1069-1086.
Wei W Z, Shi W P, Galy A, Lichlyter D, Hernandez S, Groner B, Heilbrun L, Jones R F: Protection against mammary tumor growth by vaccination with full-length, modified human ErbB-2 DNA. Int J Cancer 1999, 81 (5): 748-754.
Welling G W, Weijer W J, van der Zee R, Welling-Wester S: Prediction of sequential antigenic regions in proteins. FEBS letters 1985, 188 (2): 215-218.
Xu H, Tan P, Zheng X, Huang Y, Lin T, Wei Q, Ai J, Yang L: Immune-related adverse events following administration of anti-cytotoxic T-lymphocyte-associated protein-4 drugs: a comprehensive systematic review and meta-analysis. Drug Des Devel Ther 2019, 13:2215-2234.
Yarden Y, Sliwkowski M X: Untangling the ErbB signalling network. Nature reviews Molecular cell biology 2001, 2 (2): 127-137.
Zak K M, Kitel R, Przetocka S, Golik P, Guzik K, Musielak B, Domling A, Dubin G, Holak T A: Structure of the Complex of Human Programmed Death 1, PD-1, and Its Ligand PD-L1. Structure 2015, 23 (12): 2341-2348.
Zhu Z, Witte L: Inhibition of tumor growth and metastasis by targeting tumor-associated angiogenesis with antagonists to the receptors of vascular endothelial growth factor. Investigational new drugs 1999, 17 (3): 195-212.

F. Sequences

SEQ ID NO: 1 human CTLA residues 1-223
MACLGFQRHKAQLNLATRTWPCTLLFFLLFIPVFCKAMHVAQPAVVLASSRGIASFVCEY
ASPGKATEVRVTVLRQADSQVTEVCAATYMMGNELTFLDDSICTGTSSGNQVNLTIQGL
RAMDTGLYICKVELMYPPPYYLGIGNGTQIYVIDPEPCPDSDFLLWILAAVSSGLFFYSFL
LTAVSLSKMLKKRSPLTTGVYVKMPPTEPECEKQFQPYFIPIN
SEQ ID NO: 2 CTLA-4 (59-77)
EYASPGKATEVRVTVLRQA
SEQ ID NO: 3 CTLA-4 (75-92)
RQADSQVTEVCAATYMMG
SEQ ID NO: 4 CTLA-4 (92-114)
GNELTFLDDSICTGTSSGNQVNFHMSVVRARRNDSGTYL
SEQ ID NO: 5 CTLA-4 (130-150)
KVELMYPPPYYLGIGNGTQIY
SEQ ID NO: 6 Measles virus fusion protein (MVF)
KLLSLIKGVIVHRLEGVE
SEQ ID NO: 7 Linker
GPSL
SEQ ID NO: 8 MVF-CTLA-4 (59-77)
KLLSLIKGVIVHRLEGVEGPSLEYASPGKATEVRVTVLRQA
SEQ ID NO: 9 MVF-CTLA-4 (75-92)
KLLSLIKGVIVHRLEGVEGPSLRQADSQVTEVCAATYMMG
SEQ ID NO: 10 MVF-CTLA-4 (92-114)
KLLSLIKGVIVHRLEGVEGPSLGNELTFLDDSICTGTSSGNQVNFHMSVVRARRNDSGTY
L
SEQ ID NO: 11 MVF-CTLA-4 (130-150)
KLLSLIKGVIVHRLEGVEGPSLKVELMYPPPYYLGIGNGTQIY
SEQ ID NO: 12 CTLA-4 (77-59) PEPTIDE RETRO-INVERSO
AQRLVTVRVETAKGPSAYE
SEQ ID NO: 13 CTLA-4 (92-75) PEPTIDE RETRO-INVERSO
GMMYTAACVETVQSDAQR
SEQ ID NO: 14 CTLA-4 (114-92) PEPTIDE RETRO-INVERSO
LYTGSDNRRARVVSMHFNVQNGSSTGTCISDDLFTLENG
SEQ ID NO: 15 CTLA-4 (150-130) PEPTIDE RETRO-INVERSO
YIQTGNGIGLYYPPPYMLEVK
SEQ ID NO: 16 MVF CTLA-4 (77-59) PEPTIDE RETRO-INVERSO
KLLSLIKGVIVHRLEGVEGPSLAQRLVTVRVETAKGPSAYE
SEQ ID NO: 17 MVF CTLA-4 (92-75) PEPTIDE RETRO-INVERSO
KLLSLIKGVIVHRLEGVEGPSLGMMYTAACVETVQSDAQR
SEQ ID NO: 18 MVF CTLA-4 (114-92) PEPTIDE RETRO-INVERSO
KLLSLIKGVIVHRLEGVEGPSLLYTGSDNRRARVVSMHFNVQNGSSTGTCISDDLFTLEN
G
SEQ ID NO: 19 MVF CTLA-4 (150-130) PEPTIDE RETRO-INVERSO
KLLSLIKGVIVHRLEGVEGPSLYIQTGNGIGLYYPPPYMLEVK
SEQ ID NO: 20 TT
NSVDDALINSTIYSYFPSV
SEQ ID NO: 21 TT1
PGINGKAIHLVNNQSSE
SEQ ID NO: 22 P2
QYIKANSKFIGITEL
SEQ ID NO: 23 P30
FNNFTVSFWLRVPKVSASHLE
SEQ ID NO: 24 MVF (natural)
LSEIKGVIVHRLEGV
SEQ ID NO: 25 HBV
FFLLTRILTIPQSLN
SEQ ID NO: 26 CSP
TCGVGVRVRSRVNAANKKPE
SEQ ID NO: 27 Ac-CTLA-4 (59-77)
Ac-EYASPGKATEVRVTVLRQA
SEQ ID NO: 28 Ac-CTLA-4 (75-92)
Ac-RQADSQVTEVCAATYMMG
SEQ ID NO: 29 Ac-CTLA-4 (92-114)
Ac-GNELTFLDDSICTGTSSGNQVNFHMSVVRARRNDSGTYL
SEQ ID NO: 30 Ac-CTLA-4 (130-150)
Ac-KVELMYPPPYYLGIGNGTQIY
SEQ ID NO: 31 Ac-CTLA-4 (77-59) PEPTIDE RETRO-INVERSO
Ac-AQRLVTVRVETAKGPSAYE
SEQ ID NO: 32 Ac-CTLA-4 (92-75) PEPTIDE RETRO-INVERSO
Ac-GMMYTAACVETVQSDAQR
SEQ ID NO: 33 Ac-CTLA-4 (114-92) PEPTIDE RETRO-INVERSO
Ac-LYTGSDNRRARVVSMHFNVQNGSSTGTCISDDLFTLENG
SEQ ID NO: 34 Ac-CTLA-4 (150-130) PEPTIDE RETRO-INVERSO
Ac-YIQTGNGIGLYYPPPYMLEVK
SEQ ID NO: 35 CTLA-4 (59-77) D-amino acid all residues D-enantiomer
EYASPGKATEVRVTVLRQA
SEQ ID NO: 36 CTLA-4 (75-92) D-amino acid all residues D-enantiomer
RQADSQVTEVCAATYMMG
SEQ ID NO: 37 CTLA-4 (92-114) D-amino acid all residues D-enantiomer
GNELTFLDDSICTGTSSGNQVNFHMSVVRARRNDSGTYL
SEQ ID NO: 38 CTLA-4 (130-150) D-amino acid all residues D-enantiomer
KVELMYPPPYYLGIGNGTQIY
SEQ ID NO: 39 Ac-CTLA-4 (59-77) D-amino acid all residues D-enantiomer
Ac-EYASPGKATEVRVTVLRQA
SEQ ID NO: 40 Ac-CTLA-4 (75-92) D-amino acid all residues D-enantiomer
Ac-RQADSQVTEVCAATYMMG
SEQ ID NO: 41 Ac-CTLA-4 (92-114) D-amino acid all residues D-enantiomer
Ac-GNELTFLDDSICTGTSSGNQVNFHMSVVRARRNDSGTYL
SEQ ID NO: 42 Ac-CTLA-4 (130-150) D-amino acid all residues D-enantiomer
Ac-KVELMYPPPYYLGIGNGTQIY
SEQ ID NO: 43 MVF-CTLA-4 (59-77) D-amino acid residues 1-22 are L-amino
acids and 23-41 are D enantiomer
KLLSLIKGVIVHRLEGVEGPSLEYASPGKATEVRVTVLRQA
SEQ ID NO: 44 MVF-CTLA-4 (75-92) D-amino acid residues 1-22 are L-amino
acids and 23-40 are D enantiomer
KLLSLIKGVIVHRLEGVEGPSLRQADSQVTEVCAATYMMG
SEQ ID NO: 45 MVF-CTLA-4 (92-114) D-amino acid residues 1-22 are L-amino
acids and 23-61 are D enantiomer
KLLSLIKGVIVHRLEGVEGPSLGNELTFLDDSICTGTSSGNQVNFHMSVVRARRNDSGTY
L
SEQ ID NO: 46 MVF-CTLA-4 (130-150) D-amino acid residues 1-22 are L-amino
acids and 23-43 are D enantiomer
KLLSLIKGVIVHRLEGVEGPSLKVELMYPPPYYLGIGNGTQIY
SEQ ID NO: 47 CTLA-4 (77-59) PEPTIDE RETRO-INVERSO D-amino acid all
residues D-enantiomer
AQRLVTVRVETAKGPSAYE
SEQ ID NO: 48 CTLA-4 (92-75) PEPTIDE RETRO-INVERSO D-amino acid all
residues D-enantiomer
GMMYTAACVETVQSDAQR
SEQ ID NO: 49 CTLA-4 (114-92) PEPTIDE RETRO-INVERSO D-amino acid all
residues D-enantiomer
LYTGSDNRRARVVSMHFNVQNGSSTGTCISDDLFTLENG
SEQ ID NO: 50 CTLA-4 (150-130) PEPTIDE RETRO-INVERSO D-amino acid all
residues D-enantiomer
YIQTGNGIGLYYPPPYMLEVK
SEQ ID NO: 51 Ac-CTLA-4 (77-59) PEPTIDE RETRO-INVERSO D-amino acid all
residues D-enantiomer
Ac-AQRLVTVRVETAKGPSAYE
SEQ ID NO: 52 Ac-CTLA-4 (92-75) PEPTIDE RETRO-INVERSO D-amino acid all
residues D-enantiomer
Ac-GMMYTAACVETVQSDAQR
SEQ ID NO: 53 Ac-CTLA-4 (114-92) PEPTIDE RETRO-INVERSO D-amino acid all
residues D-enantiomer
Ac-LYTGSDNRRARVVSMHFNVQNGSSTGTCISDDLFTLENG
SEQ ID NO: 54 Ac-CTLA-4 (150-130) PEPTIDE RETRO-INVERSO D-amino acid all
residues D-enantiomer
Ac-YIQTGNGIGLYYPPPYMLEVK
SEQ ID NO: 55 MVF CTLA-4 (77-59) PEPTIDE RETRO-INVERSO D-amino acid
residues 1-22 are L-amino acids and 23-41 are D enantiomer
KLLSLIKGVIVHRLEGVEGPSLAQRLVTVRVETAKGPSAYE
SEQ ID NO: 56 MVF CTLA-4 (92-75) PEPTIDE RETRO-INVERSO D-amino acid
residues 1-22 are L-amino acids and 23-40 are D enantiomer
KLLSLIKGVIVHRLEGVEGPSLGMMYTAACVETVQSDAQR
SEQ ID NO: 57 MVF CTLA-4 (114-92) PEPTIDE RETRO-INVERSO D-amino acid
residues 1-22 are L-amino acids and 23-61 are D enantiomer
KLLSLIKGVIVHRLEGVEGPSLLYTGSDNRRARVVSMHFNVQNGSSTGTCISDDLFTLEN
G
SEQ ID NO: 58 MVF CTLA-4 (150-130) PEPTIDE RETRO-INVERSO D-amino acid
residues 1-22 are L-amino acids and 23-43 are D enantiomer
KLLSLIKGVIVHRLEGVEGPSLYIQTGNGIGLYYPPPYMLEVK

1. A cytotoxic T-lymphocyte-associated protein 4 (CTLA-4) chimeric peptide for stimulating an immune response to a CTLA-4 protein comprising one or more CTLA-4 B cell epitopes, a T helper (Th) epitope, and a linker joining the CTLA-4 B cell epitope to the Th epitope, wherein the one or more CTLA-4 B cell epitopes consist of a sequence selected from the group consisting of SEQ ID NO: 2, SEQ ID NO: 3, SEQ ID NO: 4, and SEQ ID NO: 5 or a synthetic CTLA-4 peptide for stimulating an immune response to a CTLA-4 protein comprising one or more of the sequences as set forth in SEQ ID NO: 12, SEQ ID NO: 13, SEQ ID NO: 14, or SEQ ID NO: 15.

2. The chimeric peptide of claim 1, wherein the Th epitope comprises a measles virus fusion protein peptide.

3. The chimeric peptide of claim 1, wherein the Th epitope comprises SEQ ID NO: 6.

4. The chimeric peptide of claim 1, wherein the linker comprises SEQ ID NO: 7.

5. The chimeric peptide of claim 1, wherein the peptide comprises the amino acid sequence as set forth in SEQ ID NO: 8, SEQI DNO: 9, SEQ ID NO: 10 or SEQ ID NO:11.

6. The chimeric peptide of claim 1, wherein the amino acids comprising the synthetic CTLA-4 peptide are the D enantiomer.

7. The chimeric peptide of claim 6, wherein the peptide comprises the amino acid sequence as set forth in SEQ ID NO: 43, SEQI DNO: 44, SEQ ID NO:45 or SEQ ID NO:46.

8. (canceled)

9. A chimeric peptide comprising one or more synthetic peptides of claim 1, further comprising a Th epitope, and a linker joining the synthetic CTLA-4 peptide to the Th epitope.

10. The chimeric peptide of claim 9, wherein the Th epitope comprises a measles virus fusion protein peptide.

11. The chimeric peptide of claim 9, wherein the Th epitope comprises SEQ ID NO: 6 and/or the linker comprises SEQ ID NO: 7.

12. (canceled)

13. The chimeric peptide of claim 9, wherein the peptide comprises the amino acid sequence as set forth in SEQ ID NO: 16, SEQ ID NO: 17, SEQ ID NO: 18, or SEQ ID NO: 19.

14. The or chimeric peptide of claim 1, wherein the amino acids comprising the synthetic CTLA-4 peptide are the D enantiomer.

15. The synthetic or chimeric peptide of claim 14, wherein the peptide comprises the amino acid sequence as set forth in SEQ ID NO: 47, SEQ ID NO: 48, SEQ ID NO: 49, SEQ ID NO: 50, SEQ ID NO: 55, SEQ ID NO: 56, SEQ ID NO: 57, or SEQ ID NO: 58.

16. The chimeric or synthetic peptide of claim 1, wherein the peptide is acetylated.

17. (canceled)

18. The chimeric or synthetic peptide of claim 16, wherein the peptide comprises the amino acid sequence as set forth in SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 39, SEQ ID NO: 40, SEQ ID NO: 41, SEQ ID NO: 42, SEQ ID NO: 51, SEQ ID NO: 52, SEQ ID NO: 53, and SEQ ID NO: 54.

19. A pharmaceutical composition comprising one or more chimeric peptides or synthetic peptides of claim 1 and a pharmaceutically acceptable vehicle.

20. The pharmaceutical composition of claim 19, wherein the vehicle is biodegradable and is selected from the group consisting of an emulsion comprising a pharmaceutically acceptable oil/water emulsion and a biodegradable microsphere or nanosphere comprising a polylactide-polyglycolic acid polymer.

21. An antibody that specifically binds to any of the chimeric or synthetic peptides of claim 1.

22. A method of treating a cancer, Alzheimer's disease, or autoimmune disease in a subject comprising administering to the subject any of the chimeric peptides or synthetic peptides of claim 1.

23. A method of treating a cancer, Alzheimer's disease, or an autoimmune disease in a subject comprising administering to a subject a CTLA-4 chimeric peptide wherein the chimeric peptide comprises one or more CTLA-4 B cell epitopes, a T helper (Th) epitope, and a linker joining the CTLA-4 B cell epitope to the Th epitope, wherein the one or more CTLA-4 B cell epitopes consist of a sequence selected from the group consisting of SEQ ID NO: 2, SEQ ID NO: 3, SEQ ID NO: 4, SEQ ID NO: 5, SEQ ID NO: 12, SEQ ID NO: 13, SEQ ID NO: 14, SEQ ID NO: 15, SEQ ID NO: 35, SEQ ID NO: 36, SEQ ID NO: 37, SEQ ID NO: 38, SEQ ID NO: 47, SEQ ID NO: 48, SEQ ID NO: 49, or SEQ ID NO: 50.

24. (canceled)

25. A method of treating a cancer, Alzheimer's disease, or autoimmune disease in a subject comprising administering to a subject a CTLA-4 synthetic peptide wherein the CTLA-4 synthetic peptide comprises one or more of the sequences as set forth in SEQ ID NO: 2, SEQ ID NO: 3, SEQ ID NO: 4, SEQ ID NO: 5, SEQ ID NO: 12, SEQ ID NO: 13, SEQ ID NO: 14, or SEQ ID NO: 15, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 47, SEQ ID NO: 48, SEQ ID NO: 49, or SEQ ID NO: 50.

26. (canceled)

27. (canceled)
